(19) United States
(12) Patent Application Publication
(10) Pub. No.: US 2018/0147380 A1
(43) Pub. Date: May 31, 2018

(54) APPARATUS AND METHOD FOR FORMING AN OPENING IN PATIENT'S TISSUE

(71) Applicant: Hansa Medical Products, Inc., Carmel, IN (US)

(72) Inventors: Eric D. Blom, Carmel, IN (US); Dart A. Fox, Indianapolis, IN (US); Brian Kamradt, Indianapolis, IN (US)

(21) Appl. No.: 15/878,181

(22) Filed: Jan. 23, 2018

Related U.S. Application Data
(63) Continuation of application No. 14/996,426, filed on Jan. 15, 2016.
(60) Provisional application No. 62/103,842, filed on Jan. 15, 2015.
(60) Provisional application No. 62/115,097, filed on Feb. 11, 2015.
(60) Provisional application No. 62/118,674, filed on Feb. 20, 2015.

(51) Int. Cl. A61M 16/04; A61B 90/00; A61B 17/34; A61M 25/10; A61M 16/00; A61B 17/00

(52) U.S. Cl. CPC A61M 16/0472; A61B 90/06; A61B 17/3415; A61M 25/10; A61B 2017/00039; A61B 17/3494; A61B 2017/00084; A61M 2205/6063; A61M 16/0003

(57) ABSTRACT

A surgical instrument system for use in a surgical procedure is disclosed. The surgical instrument system may include an instrument configured to puncture the tissue of a patient and detect when the instrument has entered a lumen of the patient's body. The surgical instrument system may include a balloon catheter including a percutaneous dilation balloon and a moveably positionable retainer. The retainer is configured to move relative to the balloon such that upon inflation of the balloon when the balloon is positioned in the opening in the patient's tissue, the retainer engages the patient's tissue to inhibit movement of the balloon catheter.

This application claims priority under 35 U.S.C. § 120 to U.S. application Ser. No. 14/996,426, which was filed on Jan. 15, 2016 and claimed priority under 35 U.S.C. § 119 to provisional application 62/103,842, which was filed on Jan. 15, 2015, provisional application 62/115,097, which was filed on Feb. 11, 2015, and provisional application 62/118,674, which was filed on Feb. 20, 2015. Each of those applications is expressly incorporated herein by reference.

### TECHNICAL FIELD

The present disclosure relates to instruments for forming and dilating an opening in a patient's tissue and, more specifically, for dilating an opening through a tracheal wall of a patient.

### BACKGROUND

There are a number of techniques for establishing an adequate air passageway for a patient. When the trachea, nostrils and/or mouth are free of obstruction, endotracheal intubation, which involves the insertion of a tube through the nostrils or mouth and into the trachea itself, may be used. One endotracheal tube system for use in endotracheal intubation is described in International Patent Application Publication No. WO2014/088904, which is incorporated herein by reference.

Another technique for establishing an adequate air passageway involves the creation of a puncture or incision in the tracheal wall. A tracheostomy tube may then be inserted through the opening to form a passageway that effectively bypasses the upper trachea, nostrils and/or mouth. The initial incision may be made with a smaller needle and then enlarged or dilated to receive the tracheostomy tube.

Various techniques and devices for creating punctures or other incisions in the soft issue of a patient are illustrated and described in: “An Endoscopic Technique for Restoration of Voice After Laryngectomy,” Annals of Ontology, Rhinology And Laryngology, Singer et al., 89: 529-533, 1980, “Tracheoesophageal Puncture,” Atlas of Transnasal Esphagoscopy,” Postma, et al., 2007, Tracheoesophageal Puncture in the Office Setting with Local Anesthesia, Annals of Ontology, Rhinology And Laryngology, Desyatnikova et al., 110, 613-616, 2001, A Failsafe Technique For Tracheoesophagal Puncture, Koch, The Laryngoscope, 111, September 2001, A New Method For Tracheoesophagal Puncture Under Topical Anesthesia, Gross et al., The Laryngoscope, 104, February 1994, and the Blom-Singer® Voice Prosthesis Placement Surgical Kit available from Inhealth® Technologies. Another device for use with the soft tissue of a patient is the BD Angiocath Autoguard Shielded IV Catheter, which is commercially available from Becton, Dickinson and Company of New Jersey.

There are also the devices and methods illustrated and described in U.S. Pat. Nos. 5,653,230; 5,217,005; and 8,696,697; and U.S. Pat. App. Pub. No. 2012/0180787. The disclosures of these references are hereby incorporated herein by reference. This listing is not intended as a representation that a complete search of all relevant prior art has been conducted, or that no better references than those listed exist.

### SUMMARY

According to one aspect of the disclosure, a surgical instrument system for detecting a lumen in a patient's body is disclosed. The instrument system includes a surgical instrument that monitors changes in electrical capacitance normally present in tissue to determine when the instrument has entered the lumen. In one embodiment, the surgical instrument includes a needle extending from a handle. The entire instrument except for the cutting tip may be insulated. As the tip is advanced through tissue, the instrument detects fluctuating levels of electrical capacitance. When the tip enters, for example, an air-filled target lumen, a significant drop in electrical capacitance is detected, which the instrument associates with the entry of the tip into the lumen. When used to detect entry into other lumens of a patient's body, the device is configured to determine whether the fluctuating levels in electrical capacitance are greater than a predetermined threshold, which the instrument associates with the entry of the tip into a lumen.

When the instrument determines that the needle tip has entered the target lumen, the instrument may then activate an indicator such as, for example, a flashing light emitting diode (LED) in the instrument to alert the operator to not advance further. In one embodiment, the instrument may also be programed to instantaneously retract its tip a distance of, for example, about 8 mm. In other embodiments, the tip of the instrument may remain stationary to facilitate fluid infusion or suction. In some embodiments, the system may include a noncompliant dilation balloon on a catheter for use in procedures such as, for example, percutaneous tracheostomy or percutaneous gastrostomy. In some embodiments, the surgical instrument may be another cutting tool such as, for example, a cutting blade in which the entire blade but a portion of the cutting edge may be insulated.

In some embodiments, the instrument is configured to differentiate between different types of tissue. For example, the instrument may be configured to differentiate between the electrical capacitance associated with an internal organ and the electrical capacitance associated with cancerous tissue. In such embodiments, the instrument may activate a visual or audible in the instrument to alert the operator that the tip has exited one type of tissue and entered a different type of tissue.

According to another aspect, a method for performing a surgical procedure comprises inserting a needle tip of a surgical instrument into a patient's tissue, advancing the needle tip through the tissue, monitoring an indicator of the surgical instrument while advancing the needle tip through the tissue, and maintaining a position of the surgical instrument in response to the indicator indicating the needle tip has entered a target lumen of the patient.

Illustratively according to this aspect, the surgical instrument may be operable to automatically retract the needle tip when the needle tip has entered the target lumen of the patient.

According to another aspect, a method for performing a surgical procedure comprises energizing an indicator of a surgical instrument to provide a first indication to a user when a needle tip is engaged with a portion of patient's tissue and energizing the indicator to provide a second indication different from the first indication in response to the needle tip exiting the portion of the patient's tissue.

Illustratively according to this aspect, the method may further comprise automatically retracting the needle tip in response to the needle tip exiting the portion of the patient's tissue.

Illustratively according to this aspect, energizing the indicator to provide the second indication different from the first indication in response to the needle tip exiting the portion of the patient's tissue includes energizing the indicator when the needle tip has entered the target lumen of the patient.

Illustratively according to this aspect, energizing the indicator to provide the second indication different from the first indication in response to the needle tip exiting the portion of the patient's tissue includes energizing the indicator when the needle tip has entered another portion of the patient.

According to another aspect, a method for performing a surgical procedure comprises inserting a needle tip of a surgical instrument into a first surface of a patient's tissue, advancing the needle tip through the tissue, monitoring an indicator of the surgical instrument while advancing the needle tip through the tissue, and withdrawing the needle tip from the tissue in response to the indicator indicating the needle tip has penetrated a second surface of the tissue.

Illustratively according to this aspect, the method comprises energizing a light source of the indicator prior to inserting the needle tip into the first surface. Further according to this aspect, inserting the needle tip of the surgical instrument into the first surface of the tissue comprises closing an electrical switch to de-energize the light source.

Illustratively according to this aspect, closing the electrical switch includes engaging an electrical contact with the tissue to couple a contact of the electrical switch with an electrically conductive component of the surgical instrument.

Illustratively according to this aspect, the method comprises advancing a body including an electrically conductive surface into an internal passageway of the patient, whereupon the needle tip contacts the electrically conductive component to energize a light source of the indicator.

Illustratively according to this aspect, advancing the needle tip through the tissue includes advancing the needle tip from a posterior wall of a patient's trachea through an anterior wall of the patient's esophagus.

Illustratively according to this aspect, the indicator indicates when the needle tip is positioned between the anterior wall of the patient's esophagus and a posterior wall of the patient's esophagus.

Illustratively according to this aspect, advancing the needle tip through the tissue includes advancing the needle tip through a patient's skin into a patient's trachea.

Illustratively according to this aspect, the indicator indicates when the needle tip is positioned between the anterior wall of the patient's trachea and a posterior wall of the patient's trachea

Illustratively according to this aspect, advancing the needle tip through the tissue includes advancing the needle tip from a first wall of a patient's tissue into a second wall of the patient's tissue.

Illustratively according to this aspect, the indicator indicates when the needle tip is positioned between the second wall of the patient's tissue and a third wall of the patient's tissue, the second wall and the third wall cooperating to define a lumen of the patient..

According to another aspect, a surgical instrument comprises an elongated body including a needle tip configured to puncture a patient's tissue, and an indicator secured to the elongated body, the indicator being operable to provide an indication of when the needle tip has penetrated an inner surface of the tissue.

Illustratively according to this aspect, the indicator includes a light source coupled to the elongated body.

Illustratively according to this aspect, the indicator includes an electrical switch moveable between an open position in which the light source is energized, and a closed position in which the light source is de-energized.

Illustratively according to this aspect, the elongated body includes a shaft comprising an electrically conductive material, and the electrical switch includes a conductive wire extending from the needle tip. The conductive wire includes a first end that is moveable between the open position in which the first end of the conductive wire is spaced apart from the electrically conductive material, and the closed position in which the first end of the conductive wire contacts the electrically conductive material.

Illustratively according to this aspect, the switch is biased in the open position.

Illustratively according to this aspect, the elongated body includes a handle and the shaft extends from the handle to the needle tip, and the indicator includes electrical circuitry in the handle.

Illustratively according to this aspect, the shaft is a cannula.

Illustratively according to this aspect, the indicator includes an electrical switch that is moveable between a closed position in which the light source is energized, and an open position in which the light source is de-energized.

Illustratively according to this aspect, a first electrical contact of the electrical switch includes the needle tip.

Illustratively according to this aspect, the surgical instrument further comprises a target body configured to be inserted into a passageway of the patient. The target body includes an electrically conductive surface. A second electrical contact of the electrical switch includes the electrically conductive surface such that when the needle tip engages the electrically conductive surface, the switch is closed.

Illustratively according to this aspect, the elongated body includes a handle and a shaft extending distally away from the handle to the needle tip, and the indicator includes electrical circuitry positioned in the handle.

According to another aspect, a surgical instrument comprises an elongated body including a handle and a shaft extending from the handle to a distal tip configured to puncture a patient's tissue, and an indicator including a light source in the handle. The light source is de-energized when the tip is located in the tissue between an external surface of the tissue and an internal surface of the tissue.

Illustratively according to this aspect, the shaft is electrically-conductive, and the indicator includes a conductor that is moveable between a position in which the conductor is spaced apart from the elongated body and the light source is energized, and a closed position in which the conductor engages the elongated body and the light source is de-energized.

According to another aspect, a surgical instrument comprises an elongated body including a handle and a shaft extending distally away from the handle to a needle tip configured to puncture a patient's tissue, and an indicator including a light source in the handle and a target body configured to be inserted into a passageway of the patient. The indicator is operable to energize the light source when the needle tip engages the target body.

According to another aspect, a surgical instrument comprises an elongated body including a handle and a shaft extending from the handle to a distal tip configured to puncture a patient's tissue, an indicator including a light source in the handle, and a capacitive sensor. The capacitive sensor is operable to energize the light source when the distal tip is located the tissue between an external surface of the tissue and an internal surface of the tissue.

According to another aspect, a surgical instrument comprises an elongated body including a handle and a shaft extending from the handle to a distal tip configured to puncture a patient's tissue, an indicator including a light source in the handle, and an electromagnetic sensor. The electromagnetic sensor is operable to energize the light source when the distal tip is located in the tissue between an external surface of the tissue and an internal surface of the tissue.

According to another aspect, a surgical instrument comprises an elongated body including a handle and a shaft extending from the handle to a distal tip configured to puncture a patient's tissue, an indicator including a light source in the handle, and a fiber optical thermometer needle sensor. The fiber optical thermometer needle sensor is operable to energize the light source when the distal tip is located in the tissue between an external surface of the tissue and an internal surface of the tissue.

According to another aspect, a surgical instrument comprises an elongated body including a handle and a shaft extending from the handle to a distal tip configured to puncture a patient's tissue, an indicator including a light source in the handle, a capacitive sensor operable to energize the light source when the distal tip penetrates a lumen of the patient's body, and a retraction mechanism operable to automatically retract the distal tip after the distal tip penetrates the lumen of the patient's body.

According to another aspect, a dilation instrument system is disclosed. The dilation instrument system includes a percutaneous dilation balloon and a moveably positionable retainer. The percutaneous dilation balloon is included in a balloon catheter configured to be positioned in an opening defined in a tracheal wall of a patient. The catheter includes a sheath having a proximal end and a distal end, the balloon extending over the sheath between the proximal end and the distal end, and a deflectable retention flange secured to the distal end of the sheath. The retainer is positioned over the balloon and is configured to move relative to the balloon such that upon inflation of the balloon when the balloon is positioned in the opening in the tracheal wall, the retainer engages the tracheal wall to inhibit movement of the balloon catheter.

According to another aspect, a dilation instrument system comprises a percutaneous dilation balloon, a stationary deflectable retention flange, and a moveably positionable retainer. The retainer is positioned over the balloon and is configured to move relative to the balloon such that upon inflation of the balloon when the balloon is positioned in an opening in a wall of the patient's tissue, the retainer is positioned adjacent to the wall to inhibit movement of the balloon catheter.

In some embodiments, the inflatable balloon may have a maximum diameter when inflated, and the retainer may include an annular body having an inner diameter that is less than the maximum diameter of the inflatable balloon. Additionally, in some embodiments, the annular body may include a first collar extending in a first direction, a second collar extending outwardly in a second direction opposite the first direction, and a passageway extending between an opening defined in the first collar and an opening defined in the second collar. The passageway may define the inner diameter of the annular body.

In some embodiments, the retainer may be formed from an elastomeric material. Additionally, in some embodiments, the elastomeric material may be a liquid silicone rubber. The elastomeric material may have a hardness in a range of 60 and 80 durometer (all hardnesses specified herein are Shore A unless otherwise specified).

In some embodiments, the balloon may be formed from a polymeric material. Additionally, in some embodiments, the polymeric material may be selected from a group consisting of polyethylene, polyurethane, and nylon.

In some embodiments, the sheath may comprise a tip positioned at the distal end and that is formed from a first material. The sheath may comprise an elongated body extending from the tip to the proximal end. The elongated body may be formed from a second material that is harder than the first material.

In some embodiments, the dilation instrument system may further comprise a surgical instrument configured to be coupled to the balloon catheter. The surgical instrument may comprise an elongated shaft sized to be positioned in a lumen defined in the sheath and a needle tip configured to puncture the tracheal wall. The needle tip may be configured to extend outwardly from the distal end of the sheath when the surgical instrument is coupled to the balloon catheter.

In some embodiments, the surgical instrument of the dilation instrument system may further comprise a handle coupled to the elongated shaft, an indicator including a light source in the handle, and a capacitive sensor operable to energize the light source when the needle tip penetrates a lumen of the patient's trachea.

In some embodiments, the surgical instrument of the dilation instrument system may comprise a retraction mechanism operable to automatically retract the needle tip after the needle tip penetrates the lumen of the patient's trachea.

According to another aspect, a surgical instrument system comprising a catheter having a lumen defined therein, the catheter further including a distal tip formed from a first material and an elongated body extending from the distal tip to an opposite proximal end, the elongated body being formed from a second material that has a hardness greater than the first material.

Additionally, in some embodiments, the first material may have a hardness in a range of 30 durometer to 50 durometer. In some embodiments, the elongated body may include a flared section at the proximal end.

According to another aspect, a method of dilating an opening in a patient's tissue is disclosed. The method comprises advancing a distal end of a balloon catheter in a first direction through the opening in the patient's tissue, pulling the balloon catheter in a second direction opposite the first direction to engage a retention flange secured to the distal end with an inner surface of the patient's tissue, advancing a retainer along the balloon catheter in the first direction to engage an outer surface of the patient's tissue opposite the inner surface, and inflating a balloon of the balloon catheter to dilate the opening in the patient's tissue.

According to another aspect, a method of dilating an opening in a patient's tissue comprises positioning an uninflated dilation balloon in the opening in the patient's tissue, engaging a retention flange with an inner surface of the patient's tissue, advancing a moveable retainer along the balloon to a position adjacent to an outer surface of the patient's tissue opposite the inner surface, and inflating the balloon to dilate the opening in the patient's tissue.

In some embodiments, the method may further comprise positioning an elongated shaft of a surgical instrument in a lumen defined in the balloon catheter such that a needle tip of the surgical instrument extends outwardly from the distal end of the balloon catheter, inserting the needle tip of the surgical instrument into the outer surface of the patient's tissue, and advancing the needle tip through the tissue to define the opening.

In some embodiments, the surgical instrument may be operable to automatically retract the needle tip into the lumen of the balloon catheter when the needle tip has penetrated the inner surface of the tissue.

In some embodiments, the method may further comprise monitoring an indicator of the surgical instrument while advancing the needle tip through the tissue. The surgical instrument may be operable to automatically retract the needle tip in response to the indicator indicating the needle tip has penetrated the inner surface of the tissue. Additionally, in some embodiments, the indicator may be operable to provide a visual indication when the needle tip has penetrated the inner surface of the tissue.

In some embodiments, advancing the retainer along the balloon catheter in the first direction may include engaging an annular body of the retainer with the outer surface of the tissue.

According to another aspect, a dilation instrument system is disclosed. The system comprises a balloon catheter configured to be positioned in an opening defined in a patient's tissue. The catheter includes a sheath having a proximal end and an elastomeric distal end, an inflatable balloon extending over the sheath between the proximal end and the distal end, and a deformable retention flange secured to the distal end of the sheath. The system also includes a retainer positioned over the balloon and configured to move relative to the balloon such that upon inflation of the balloon when the balloon is positioned in the opening in the patient's tissue, the retainer engages the patient's tissue to inhibit movement of the balloon catheter. The system also includes a surgical instrument removably coupled to the sheath. The surgical instrument comprises a needle tip extending outwardly from the sheath that is configured to puncture the patient's tissue. The surgical instrument may further comprise a retraction mechanism operable to automatically retract the needle tip after the needle tip penetrates a lumen of the patient's tissue. Additionally, the sheath may comprise a tip positioned at the distal end that is formed from a first material, and an elongated body extending from the tip to the proximal end of the sheath. The elongated body may be formed from a second material that is harder than the first material, and the retraction mechanism is operable to retract the needle tip into the tip of the sheath.

According to another aspect, a surgical instrument system comprises an elongated body including a handle and a shaft extending from the handle to a distal end configured to pass through a patient's tissue, an indicator including a light source, a sensor operable to generate an electrical signal, and a control circuit. The control circuit is configured to receive the electrical signal from the sensor, determine whether the distal end has penetrated a lumen of a patient, and energize the light source when the distal end has penetrated the lumen of the patient.

In some embodiments, the control circuit may be configured to determine whether the distal end has engaged tissue of a patient, energize the light source in a first state when the distal end has engaged tissue of the patient, and energize the light source in a second state when the distal end has penetrated the lumen of the patient. The second state may be different from the first state. Additionally, when the light source is in the first state, the light source may be flashing.

In some embodiments, the system may further comprise a retraction mechanism operable to retract the distal end. The control circuit may be configured to energize the retraction mechanism when the distal end has penetrated the lumen of the patient.

In some embodiments, the retraction mechanism may include a biasing element configured to bias the distal end in a retracted position. In some embodiments, the retraction mechanism may include a locking arm configured to maintain the distal end in an extended position.

The retraction mechanism may include an electric motor configured to move the locking arm to a disengaged position when the electric motor is energized. When the locking arm is in the disengaged position, the biasing element may be configured to urge the distal end to move to the retracted position.

According to another aspect, a surgical instrument system configured to perform any of the method described herein is disclosed.

### BRIEF DESCRIPTION OF THE DRAWINGS

The detailed description particularly refers to the following figures, in which:

FIGS. 1A and B are a perspective view of one embodiment of a surgical instrument system for use in performing a surgical procedure and an enlarged perspective view of a detail of FIG. 1A;

FIG. 2 illustrates a circuit diagram of an electrical circuit of the surgical instrument system of FIG. 1;

FIG. 3 illustrates a sectional side elevation view of the surgical instrument system of FIG. 1 forming a puncture in tissue of a patient;

FIG. 4 illustrates a view similar to FIG. 3 showing a distal end of the surgical instrument system having reached a passageway beyond the tissue;

FIG. 5 illustrates a perspective view of another embodiment of a surgical instrument system;

FIG. 6 illustrates a circuit diagram of an electrical circuit of the surgical instrument system of FIG. 5;

FIG. 7 illustrates a cross sectional side elevation view of the surgical instrument system of FIG. 5 forming a puncture in the soft tissue of a patient;

FIG. 8 illustrates a view similar to FIG. 7 showing a distal end of the surgical instrument system having reached a passageway beyond the tissue;

FIG. 9A is a perspective view of another embodiment of a surgical instrument system for use in performing a surgical procedure;

FIG. 9B is an enlarged cross sectional view of a detail of the surgical instrument system of FIG. 9A;

FIG. 10 illustrates a circuit diagram of an electrical circuit of the surgical instrument system of FIG. 9;

FIGS. 11A and B are a perspective view of another embodiment of a surgical instrument system for use in performing a surgical procedure and an enlarged perspective view of a detail of FIG. 11A;

FIG. 12A is a perspective view of another embodiment of a surgical instrument system for use in performing a surgical procedure;

FIG. 12B is an enlarged cross sectional view of a detail of the surgical instrument system of FIG. 9A;

FIG. 13 illustrates a circuit diagram of an electrical circuit of the surgical instrument system of FIG. 12;

FIG. 14 is a perspective view illustrating a surgical instrument system for forming and dilating an opening in a patient's tissue;

FIG. 15 is a perspective view illustrating a balloon catheter and a retainer of the instrument system of FIG. 14;

FIG. 16 is a view similar to FIG. 15 illustrating the balloon catheter with the balloon inflated;

FIG. 17 is a side elevation view of a sheath of the balloon catheter of FIG. 15;

FIG. 18 is a cross-sectional elevation view of the balloon catheter taken along the line 18-18 in FIG. 16 with the retainer removed;

FIG. 19 is a partial cross-sectional plan view of a surgical instrument of the instrument system of FIG. 14;

FIG. 20 is an enlarged cross sectional view of a detail of FIG. 14;

FIG. 21 illustrates the needle retraction mechanism of the surgical instrument of FIG. 14;

FIG. 22 illustrates the needle retraction mechanism of FIG. 21 in an extended position;

FIG. 23 illustrates a circuit diagram of an electrical circuit of the surgical instrument system of FIG. 14;

FIG. 24 illustrates a perspective view of another embodiment of a surgical instrument system for use in performing a surgical procedure;

FIG. 25 illustrates a perspective view of some of the components of the system of FIG. 24; and

FIG. 26 illustrates a circuit diagram of an electrical circuit of the surgical instrument system of FIG. 24.

### DETAILED DESCRIPTION OF THE DRAWINGS

While the concepts of the present disclosure are susceptible to various modifications and alternative forms, specific exemplary embodiments thereof have been illustrated by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

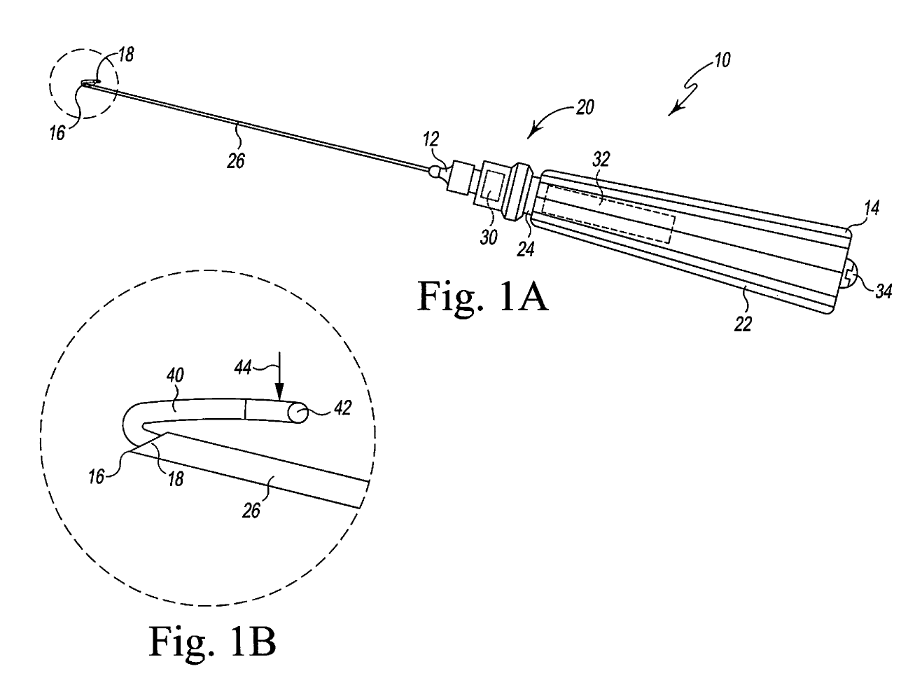

Referring now to FIGS. 1A-B, a surgical instrument system for detecting a lumen in a patient's body is illustrated. The system 10 includes a surgical instrument 10 that is configured for insertion into the soft tissue of a patient. Illustratively, the surgical instrument 10 may be use to form a puncture between the skin of the neck and the anterior wall of the trachea of a patient, but it should be appreciated that the surgical instrument 10 may be used to form other punctures, incisions, or openings in the patient's tissue. As illustrated in FIGS. 1A-B, the surgical instrument 10 includes an elongated body 12 having a proximal end 14 and a distal end 16. A needle tip 18 configured to pierce the tissue is formed at the distal end 16 of the body 12. As described in greater detail below, the surgical instrument 10 also includes an indicator 20 configured to notify a user that the needle tip 18 has penetrated the tissue.

The elongated body 12 includes a handle 22 extending from the proximal end 14 to a distal handle end 24. A shaft 26 extends distally away from the handle 22 to the needle tip 18. In the illustrated embodiment, the shaft 26 is a cannula formed from an electrically conductive material. The needle tip 18 and the shaft 26 are integral, but it should be appreciated that in other embodiments the needle tip 18 and the shaft 26 may be formed as separate components and assembled.

As illustrated in FIG. 1A, the indicator 20 is positioned at the distal handle end 24. In the illustrated embodiment, the indicator 20 includes a light source such as, for example, a light emitting diode (LED) 30, and other electrical circuitry 32 operable to energize the LED 30 to provide a visual output to the user. In other embodiments, the indicator 20 may include other electrical circuitry to provide an audible output to the user. The surgical instrument 10 also includes a power switch 34 positioned at the proximal end 14 of the handle 22. Power switch 34 is operable to supply power to the electrical circuitry 32 including LED 30.

The electrical circuitry 32 also includes a conductor 40 that extends down the shaft 26 and outwardly from the needle tip 18 to an end 42 spaced apart from the shaft 26. When sufficient force is applied in the direction indicated by arrow 44, the end 42 of the conductor 40 is pressed into contact with the shaft 26. The conductor 40 is elastic, so that when the force is removed, the conductor 40 returns to a position with the end 42 out of electrical contact with the shaft 26. In the illustrative embodiment, the conductor 40 is formed from conductive metallic spring wire.

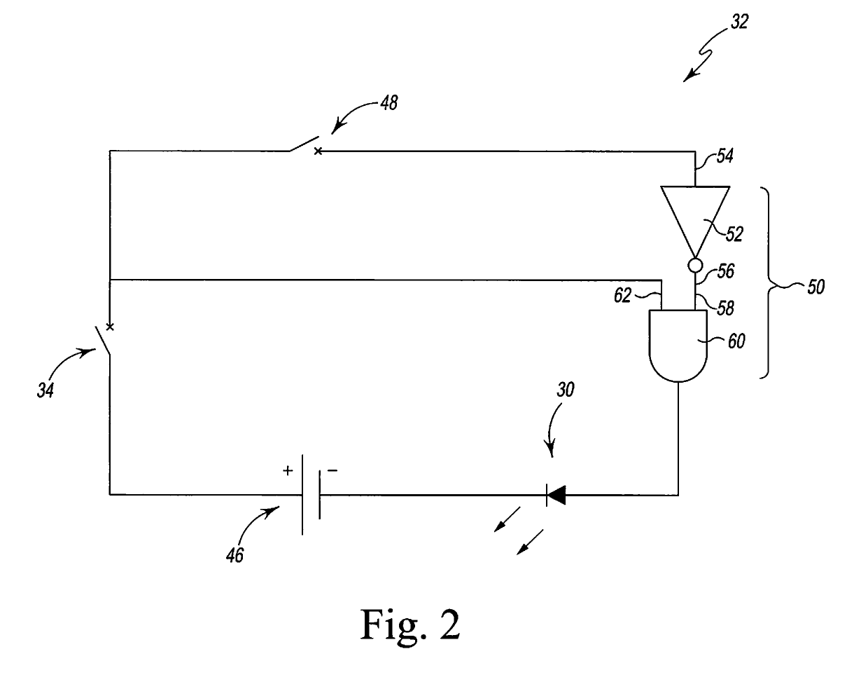

Referring now to FIG. 2, the electrical circuitry 32 includes a battery 46 configured to supply electrical power when switch 34 is closed. In the illustrative embodiment, the conductor 40 and the shaft 26 comprise the electrical contacts of an electrical switch 48 between switch 34 and a logic circuit 50 operable to control the supply of electrical power to the LED 30. The illustrative logic circuit 50 comprises an inverting amplifier and an AND gate, but the logic circuit 50 may be realized as other circuitry as appropriate.

The logic components 50 include inverting amplifier 52 configured to output an inverted version of its input. As illustrated in FIG. 2, the electrical switch 48 (i.e., the conductor 40 and the shaft 26) is coupled to the input 54 of the inverting amplifier 52. When the end 42 of the conductor 40 is spaced apart from the shaft 26, the switch 48 is open such that a zero is supplied to the input 54 of inverting amplifier 52 and TRUE is generated at the output 56 of inverting amplifier 52. When the end 42 of the conductor 40 is engaged with the shaft 26, the switch 48 is closed such that battery voltage is applied to the input 54 of inverting amplifier 52 and FALSE is generated at the output 56 of inverting amplifier 52.

The output of inverting amplifier 52 is coupled to an input 58 of an AND gate 60, the output of which energizes the LED 30 when its inputs 58, 62 are TRUE. Input 62 of the AND gate 60 is coupled through switch 34 such that when switch 34 is closed, the input 62 is at battery 46 voltage. Thus, in the illustrative embodiment, the AND gate 60 energizes the LED 30 when the switch 34 is closed and the switch 48 (between the conductor 40 and the shaft 26) is open. In other words, the LED 30 is illuminated when the switch 34 is closed and the end 42 of the conductor 40 is spaced apart from the shaft 26.

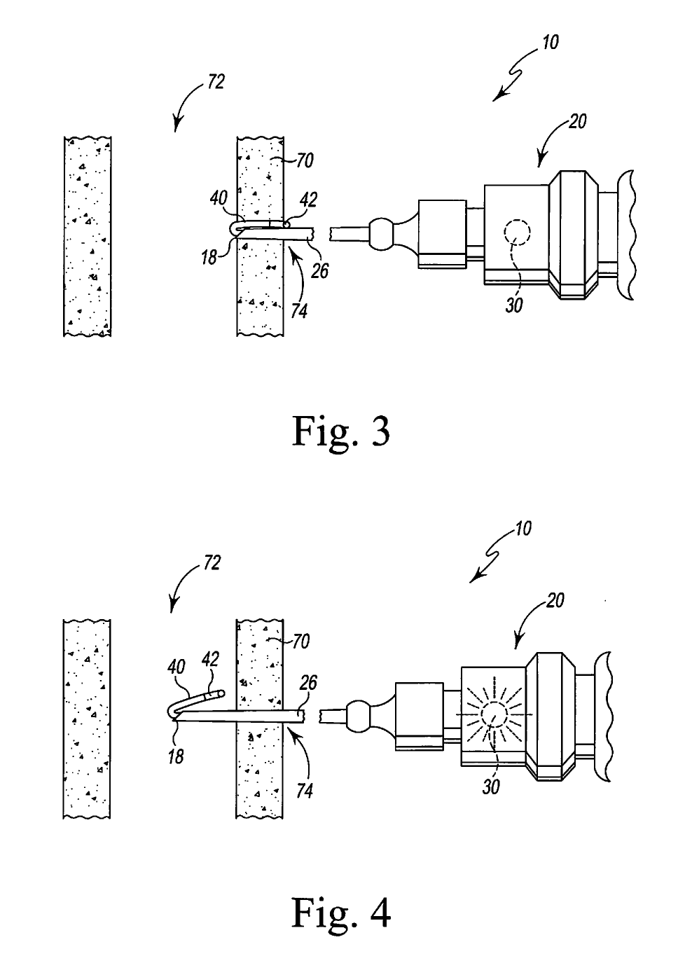

Referring now to FIGS. 3-4, some of the steps of a surgical procedure using the surgical instrument 10 are illustrated. In use, the needle tip 18 of the surgical instrument 10 may be used to form the puncture 74. With the needle tip 18 against the neck of the patient, the switch 34 is closed, turning on the LED 30. The needle tip 18 then punctures the neck. As the needle tip 18 is advanced through the neck, the tissue surrounding the needle shaft 26 causes the end 42 of the conductor 40 to engage the shaft 26, thereby closing the switch 48 and deenergizing the LED 30 as illustrated in FIG. 3.

When the needle tip 18 reaches, and protrudes into, the patient's lumen 72 (e.g., trachea, esophagus, or spinal column), the end 42 of the conductor 40 moves away from the shaft 26, as illustrated in FIG. 4. When the conductor end 42 moves away from the shaft 26, the switch 48 opens and power is again supplied to the LED 30. The LED 30 thereby provides a visual indication to the user that the instrument 10 has penetrated the patient's neck and is in the lumen 72. As noted above, in other embodiments the surgical instrument 10 may additionally or alternatively include an audible indicator to notify the user that the instrument 10 has penetrated the tissue of the patient. In response to receiving the indication that the puncture has been completed, the physician will typically withdraw the needle tip 18 from the tissue.

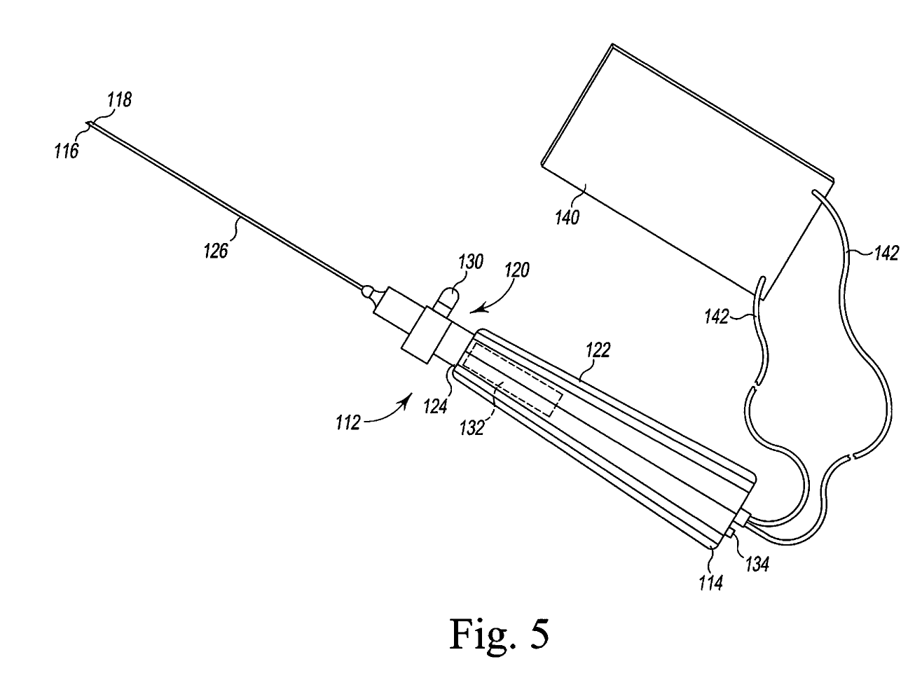

Referring now to FIGS. 5-8, another surgical instrument system including a puncture instrument 110 configured for insertion into the soft tissue of a patient is illustrated. The puncture instrument 110 includes an elongated body 112 that extends from a proximal end 114 to a distal end 116. A needle tip 118 configured to pierce the tissue is formed at the distal end 116 of the body 112. The surgical instrument 110 includes an indicator 120 configured to notify a user that the needle tip 118 has penetrated the tissue.

The elongated body 112 includes a handle 122 extending from the proximal end 114 to a distal handle end 124. A shaft 126 extends from the handle 122 to the needle tip 118. In the illustrative embodiment, the shaft 126 is a cannula formed from an electrically conductive material. The needle tip 118 and the shaft 126 are integrally formed, but it should be appreciated that in other embodiments the needle tip 118 and the shaft 126 may be formed as separate components that are assembled.

As illustrated in FIG. 5, the indicator 120 is positioned at the distal handle end 124. In the illustrative embodiment, the indicator 120 includes a light source such as, for example, an LED 130, and other electrical circuitry 132 operable to energize the LED 130 to provide a visual output to the user. In other embodiments, the indicator 120 may include other electrical circuitry to provide an audible output to the user. The surgical instrument 110 also includes a power switch 134 positioned at the proximal end 114 of the handle 122. Switch 134 is operable to supply power to the electrical circuitry 132 including LED 130.

The surgical instrument 110 also includes a target body 140 configured to be positioned in an internal lumen, for example, the esophagus, of the patient. The target body 140 comprises an electrically conductive material and is configured to be accommodated by the lumen. The target body 140 is coupled to the electrical circuitry 132 by a pair of electrical conductors 142. Illustratively, target body 140 comprises a puncture-resistant, electrically conductive balloon or the like.

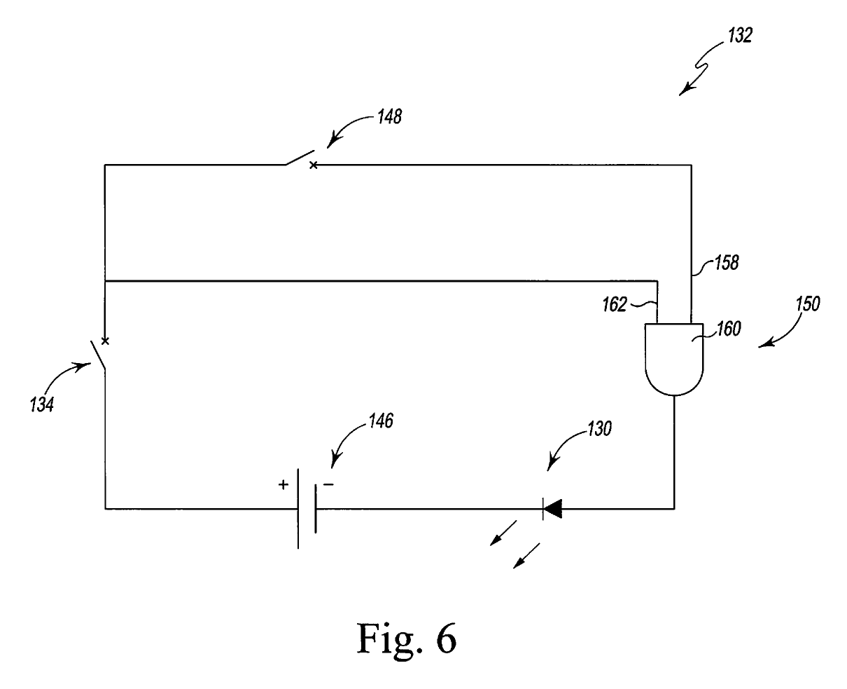

Referring now to FIG. 6, the electrical circuitry 132 includes a battery 146 configured to supply electrical power when the power switch 134 is closed. In the illustrative embodiment, the target body 140 and the needle tip 118 of the shaft 126 comprise the electrical contacts of an electrical switch 148 between switch 134 and a logic circuit 150 operable to energize LED 130. The logic circuit 150 is illustratively embodied as an AND gate, but the logic circuit 150 may be realized as other circuitry as appropriate.

Illustratively, the logic circuit 150 include a single AND gate 160 configured to energize the LED 30 when the inputs 158, 162 of AND gate 160 are high. As illustrated in FIG. 6, the input 158 of the AND gate 160 is coupled to the output of the switch 148 (comprising the target body 140 and the needle tip 118), and the input 162 of the AND gate 160 is coupled to the output of the switch 134. Thus, when both switches 134, 148 are closed, the AND gate 160 energizes the LED 130.

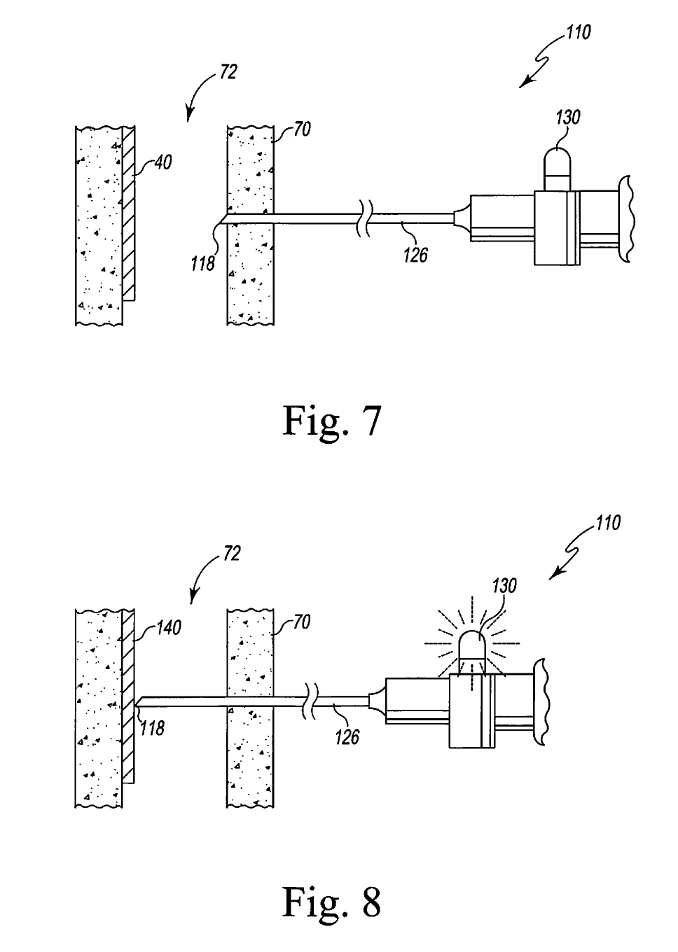

Referring now to FIGS. 7-8, some of the steps of a surgical procedure using the surgical instrument 110 are illustrated. In use, the needle tip 118 of the surgical instrument 110 may be used to form a puncture in, for example, the tracheoesophageal wall 70. Illustrated in FIG. 7, the target body 140 is introduced into the esophagus 72 to the anticipated level at which the needle tip 118 will form the puncture. With the needle tip 118 spaced apart from the tracheoesophageal wall 70, the switch 134 is closed, and the puncture commenced. The LED 130 remains deenergized because the switch 148 comprising tip 118 and target body 140 is not yet closed.

The needle tip 118 exits the tracheoesophageal wall 70 into the esophagus 72 and advances into contact with the target body 140, as illustrated in FIG. 8. When the needle tip 118 contacts the target body 140, the switch 148 closes and power is supplied to the LED 130. A visual indication to the user that the instrument 110 has penetrated the tissue of the patient and is positioned in the esophagus 72 results. As described above, in other embodiments, the surgical instrument may alternatively include an audible indicator to notify the user that the instrument has penetrated the soft tissue of the patient and is positioned in the esophagus 72. In still other embodiments, the surgical instrument may include both a visual indicator and an audible indicator. In response to receiving the indication from the LED 130, the user may withdraw the needle tip 118 from the patient's tissue.

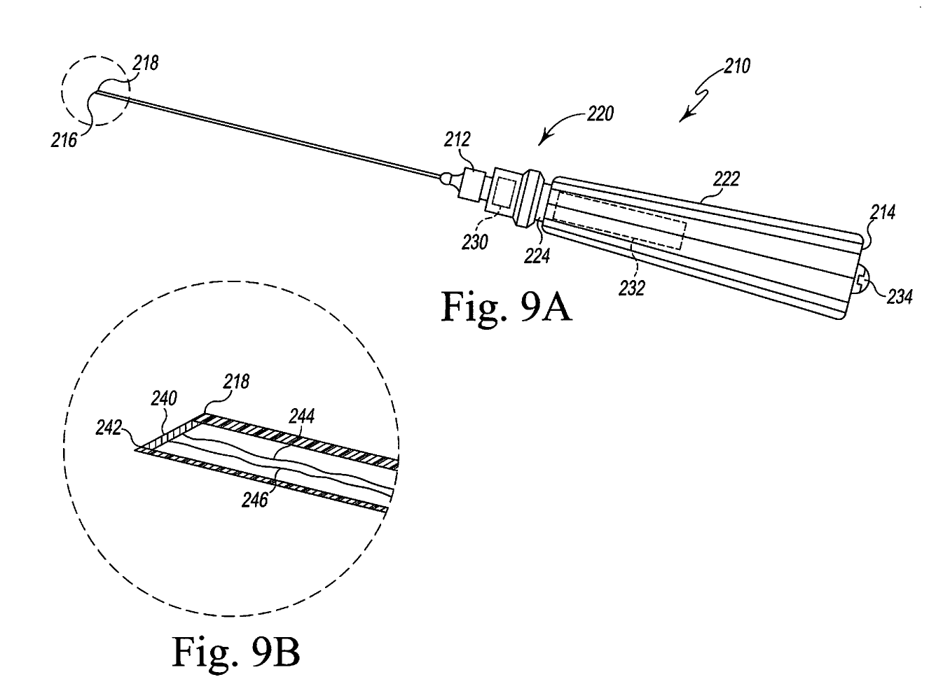

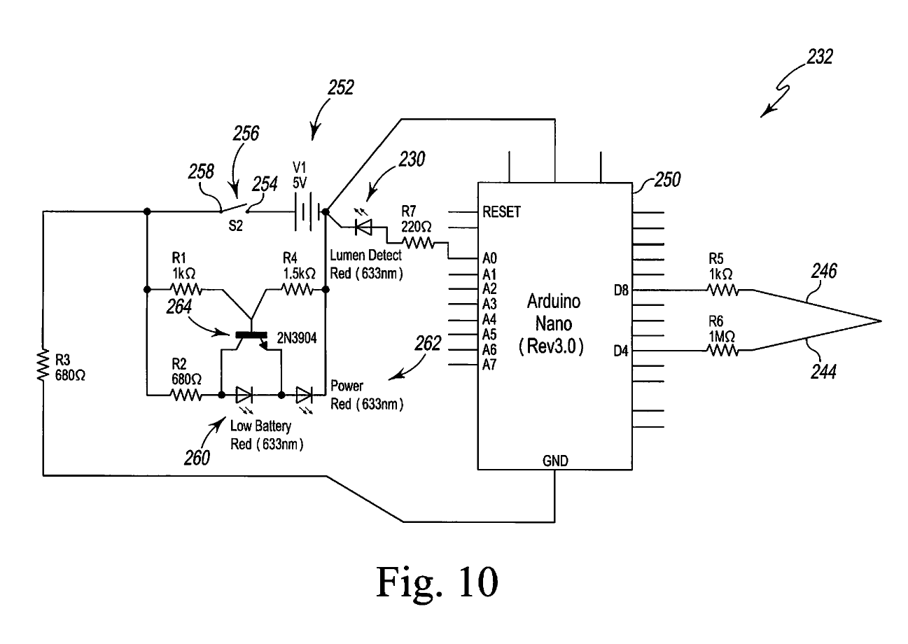

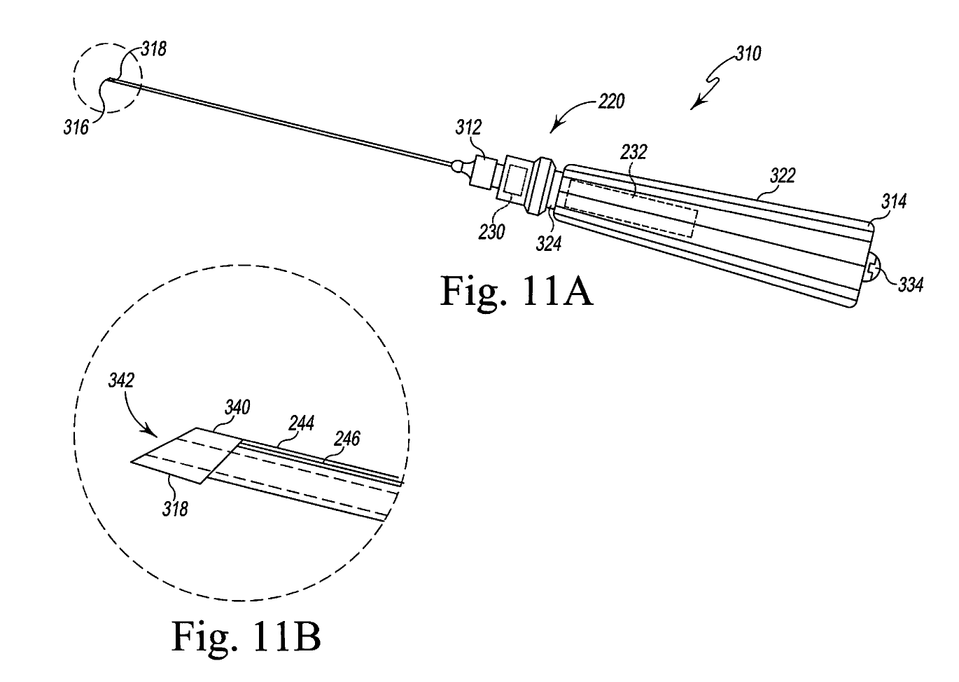

Referring now to FIGS. 9-10, another surgical instrument system including a puncture instrument 210 configured for insertion into the soft tissue of a patient is illustrated. The surgical instrument 210 includes an elongated body 212 that extends from a proximal end 214 to a distal end 216. A needle tip 218 configured to pierce the tissue is formed at the distal end 216 of the body 212. The surgical instrument 210 includes an indicator 220 configured to notify a user that the needle tip 218 has penetrated the tissue. In the illustrative embodiment, the surgical instrument 210 detects increases and decreases in capacitance and will activate the indicator 220 when the capacitance is greater than a predetermined threshold corresponding to the capacitance associated with a human body, as described in greater detail below.

The elongated body 212 includes a handle 222 extending from the proximal end 214 to a distal handle end 224. A shaft 226 extends from the handle 222 to the needle tip 218. In the illustrative embodiment, the shaft 226 is a cannula formed from a non- conductive material such as, for example, a non-conductive ceramic or plastic material. The needle tip 218 and the shaft 226 are integrally formed, but it should be appreciated that in other embodiments the needle tip 218 and the shaft 226 may be formed as separate components that are assembled.

As illustrated in FIG. 9A, the indicator 220 is positioned at the distal handle end 224. In the illustrative embodiment, the indicator 220 includes a light source such as, for example, an LED 230, and other electrical circuitry 232 operable to energize the LED 230 to provide a visual output to the user. In other embodiments, the indicator 220 may include other electrical circuitry to provide an audible output to the user. The surgical instrument 210 also includes a power switch 234 positioned at the proximal end 214 of the handle 222. Switch 234 is operable to supply power to the electrical circuitry 232 including LED 230.

As illustrated in FIG. 9B, the electrical circuitry 232 also includes a conductor plate 240 that is positioned in the distal opening 242 of the needle tip 218. In the illustrative embodiment, the plate 240 covers the opening 242 such that fluid is prevented from entering the needle tip 218. A pair of wires 244, 246 connects the backside of the plate 240 to the electrical circuitry 232. When a patient's tissue contacts the conductor plate 240, the electrical circuitry 232 is operable to detect the electrical capacitance of the tissue, as described in greater detail below.

Referring now to FIG. 10, the electrical circuitry 232 is shown in greater detail. The schematic and block circuit diagram descriptions that follow identify specific integrated circuits and other components of the circuitry 232 and in many cases specific sources for these. Specific terminal and pin names and numbers are generally given in connection with these for the purposes of completeness. It is to be understood that these terminal and pin identifiers are provided for these specifically identified components. It is to be understood that this does not constitute a representation, nor should any such representation be inferred, that the specific components, component values or sources are the only components available from the same or any other sources capable of performing the necessary functions. It is further to be understood that other suitable components available from the same or different sources may not use the same terminal/pin identifiers as those provided in this description.

The circuitry 232 includes a capacitance monitoring circuit 250, for example, an Arduino Nano (rev. 3.0) available from Arduino. A voltage supply includes a 5 VDC battery 252, the anode of which is coupled to one terminal 254 of a switch 256. The other terminal 258 of switch 256 is coupled through a 680 Ω resistor to the GrouND terminal of circuit 250. The 5V terminal of circuit 250 is coupled to the cathode of battery 252. Terminal 258 of switch 256 is coupled through a 680 Ω resistor to the anode of a red (633 nm) “Low Battery” LED 260. The cathode of LED 260 is coupled to the anode of a red “Power” LED 262. The cathode of LED 262 is coupled to the cathode of battery 252. A series voltage divider of a 1 KΩ resistor and a 1.5 KΩ resistor is coupled across terminal 258 and the cathode of battery 252. The junction of the 1 KΩ resistor and the 1.5 KΩ resistor is coupled to the base of a transistor 264, illustratively, a 2N3904 transistor. The collector of transistor 264 is coupled to the anode of LED 260. The emitter of transistor 264 is coupled to the cathode of LED 260. Thus, as long as transistor 264 is “on,” LED 260 is deenergized. When the voltage on the base of the transistor 264 drops below that required to hold it “on,” it turns off and LED 260 is energized, indicating low battery 252 voltage.

The conductor plate 240 of the needle tip 218 is coupled via the wire 244 through a 1 MΩ resistor to the D4 terminal of circuit 250. The conductor plate 240 is also coupled via the wire 246 through a 1 KΩ resistor to the D8 terminal of the circuit 250. The A0 terminal of circuit 250 is coupled through a 220 Ω resistor to the anode of a red “Lumen Detect” LED 230. The cathode of LED 230 is coupled to the cathode of battery 252. When the capacitance increases sharply, such as, for example, when the conductor plate 240 is placed in contact with a patient's tissue, the circuit 250 is programmed to switch the A0 terminal “high,” thereby turning LED 230 “on.” This indicates to the user that the needle tip 218 is engaged with the patient's tissue.

In use, the needle tip 218 of the surgical instrument 210 may be used to form a puncture in the patient's tissue. For example, with the needle tip 218 against the neck of the patient, the conductor plate 240 is engaged with the patient's tissue, and the capacitance increases sharply. The circuit 250 switches the A0 terminal “high,” thereby turning LED 230 “on.” The needle tip 218 then punctures the neck. As the needle tip 218 is advanced through the neck, the conductor plate 240 remains engaged with the patient's tissue and the LED 230 remains energized. When needle tip 218 reaches, and protrudes into, a target lumen of a patient such as, for example, the trachea, esophagus, or spinal column, the conductor plate 240 is no longer in contact with the patient's tissue, thereby causing the capacitance to decrease sharply, and the circuit 250 switches the A0 terminal “low,” thereby turning LED 230 “off.” This indicates to the user that the needle tip 218 is positioned in the target lumen.

The surgical instrument of FIGS. 9-10 utilizes the stored electrical charge or capacitance of the human body to detect when the needle is engaged with a patient's tissue. In other embodiments, the surgical instrument may employ an oscillator or other type of tuned-circuit amplifier to produce an AC current (a current that regularly switches its polarity back and forth). The oscillator may be connected to a conductor plate positioned at the needle tip, which also has the ability to store electrical charge thus has its own capacitance. When the conductor plate is engaged with a patient's tissue, the body's capacitance is introduced into the circuit, and the oscillator has to pump charge into a much larger surface area. That causes the oscillator to detune, or change frequency. This change can be monitored and used to toggle the indicator on or off.

Referring now to FIGS. 11A-B, another surgical instrument system including a puncture instrument 310 that uses the patient's capacitance to provide an indication to the user is shown. The surgical instrument 310 includes an elongated body 312 that extends from a proximal end 314 to a distal end 316. A needle tip 318 configured to pierce the tissue is formed at the distal end 316 of the body 312. The surgical instrument 310 includes an indicator 220 configured to notify a user that the needle tip 318 has penetrated the tissue. In the illustrative embodiment, the surgical instrument 310 detects increases and decreases in capacitance and will activate the indicator 220 when the capacitance is greater than a predetermined threshold corresponding to the capacitance associated with a human body.

The elongated body 312 includes a handle 322 extending from the proximal end 314 to a distal handle end 324. A shaft 326 extends from the handle 322 to the needle tip 318. In the illustrative embodiment, the shaft 326 is a cannula formed from a non- conductive material. The needle tip 318 and the shaft 326 are integrally formed, but it should be appreciated that in other embodiments the needle tip 318 and the shaft 326 may be formed as separate components that are assembled.

As illustrated in FIG. 11A, the indicator 220 is positioned at the distal handle end 324. In the illustrative embodiment, the indicator 220 includes a light source such as, for example, an LED 230, and other electrical circuitry 232 operable to energize the LED 230 to provide a visual output to the user. In other embodiments, the indicator 220 may include other electrical circuitry to provide an audible output to the user. The surgical instrument 310 also includes a power switch 334 positioned at the proximal end 314 of the handle 322. Switch 334 is operable to supply power to the electrical circuitry 332 including LED 330.

As illustrated in FIG. 11B, the electrical circuitry 232 also includes a conductor sheath 340 that is positioned over the needle tip 318. In the illustrative embodiment, the sheath 340 does not cover the opening 342 of the needle tip 318 such that fluid is permitted to enter the needle tip 318. A pair of wires 244, 246 connects the sheath 340 to the electrical circuitry 232. When a patient's tissue contacts the conductor sheath 340, the electrical circuitry 232 is operable to detect the electrical capacitance of the tissue.

The electrical circuitry 232 in the embodiment of FIGS. 11A-B is identical to the electrical circuitry described above in regard to FIGS. 9-10. In use, the needle tip 318 of the surgical instrument 310 may be used to form a puncture in the patient's tissue. With the needle tip 318 against the tissue of the patient, the conductor sheath 340 is engaged with the patient's tissue, and the capacitance increases sharply. The circuit 250 of the electrical circuitry 232 switches the A0 terminal “high,” thereby turning LED 230 “on.” The needle tip 318 then punctures the neck. As the needle tip 318 is advanced through the neck, the conductor sheath 340 remains engaged with the patient's tissue and the LED 230 remains energized. When needle tip 318 reaches, and protrudes into, the target lumen, the capacitance decreases sharply, and the circuit 250 switches the A0 terminal “low,” thereby turning LED 230 “off.” This indicates to the user that the needle tip 318 is positioned in the target lumen of the patient.

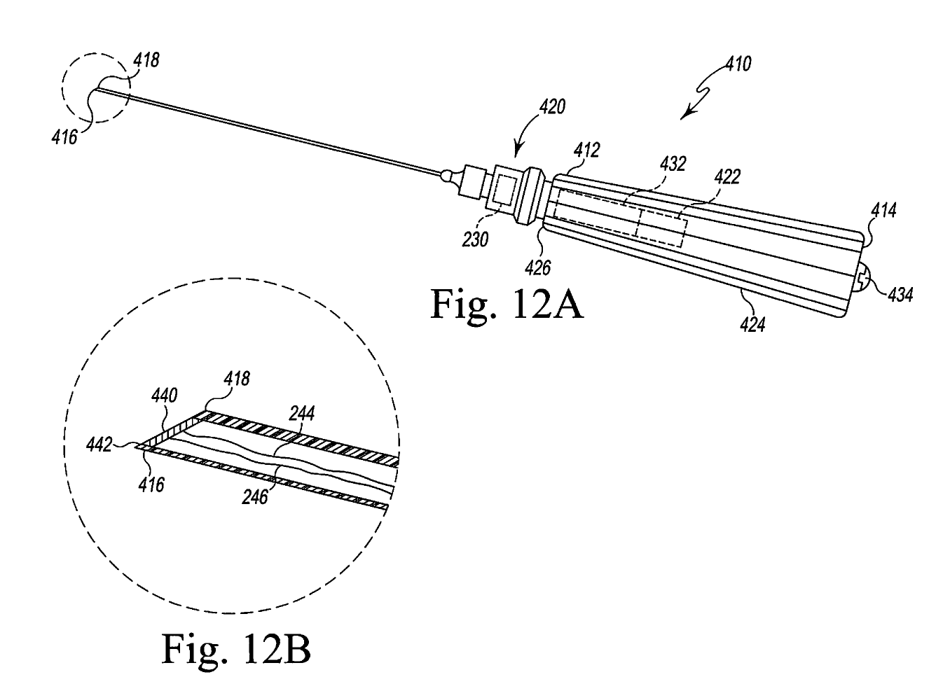

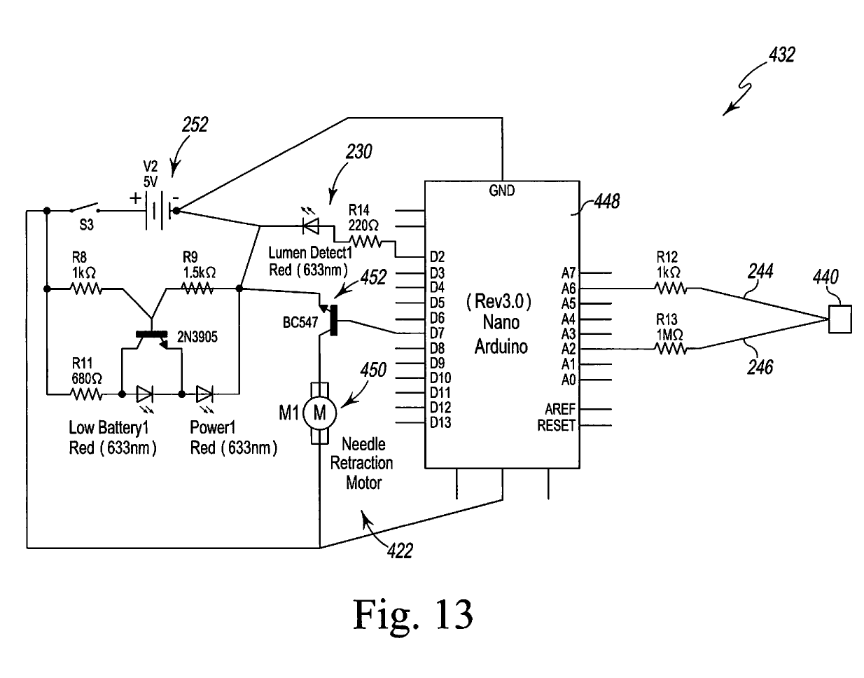

Referring now to FIGS. 12-13, another system including a surgical instrument 410 configured for insertion into the soft tissue of a patient is illustrated. The surgical instrument 410 includes an elongated body 412 that extends from a proximal end 414 to a distal end 416. A needle tip 418 configured to pierce the tissue is formed at the distal end 416 of the body 412. The surgical instrument 410 includes an indicator 420 configured to notify a user that the needle tip 418 has penetrated the tissue and an automatic needle retraction mechanism 422 operable to retract the needle tip 418 after it has penetrated the tissue, as described in greater detail below. In the illustrative embodiment, the surgical instrument 410 detects increases and decreases in capacitance and will activate the indicator 420 when the capacitance is greater than a predetermined threshold corresponding to the capacitance associated with a human body, as described in greater detail below.

The elongated body 412 includes a handle 424 extending from the proximal end 414 to a distal handle end 426. A shaft 428 extends from the handle 424 to the needle tip 418. In the illustrative embodiment, the shaft 428 is a cannula formed from a non- conductive material. The needle tip 418 and the shaft 428 are integrally formed, but it should be appreciated that in other embodiments the needle tip 418 and the shaft 428 may be formed as separate components that are assembled.

As illustrated in FIG. 12A, the indicator 420 is positioned at the distal handle end 426. In the illustrative embodiment, the indicator 420 includes a light source such as, for example, an LED 230, and other electrical circuitry 432 operable to energize the LED 230 to provide a visual output to the user. In other embodiments, the indicator 420 may include other electrical circuitry to provide an audible output to the user. The surgical instrument 410 also includes a power switch 434 positioned at the proximal end 414 of the handle 424. Switch 434 is operable to supply power to the electrical circuitry 432 including LED 230.

As illustrated in FIG. 12B, the electrical circuitry 432 also includes a conductor plate 440 that is positioned in the distal opening 442 of the needle tip 418. In the illustrative embodiment, the plate 440 covers the opening 442 such that fluid is prevented from entering the needle tip 418. A pair of wires 244, 246 connects the backside of the plate 440 to the electrical circuitry 432. When a patient's tissue contacts the conductor plate 440, the electrical circuitry 432 is operable to detect the electrical capacitance of the tissue, as described in greater detail below.

Referring now to FIG. 13, the electrical circuitry 432 is shown in greater detail. Except as otherwise discussed in the description that follows, the circuitry 432 illustrated in FIG. 13 is identical to the circuitry 232 illustrated in FIG. 10. The electrical circuitry 432 includes a circuit 448 that is operable to activate the indicator 420 and the automatic needle retraction mechanism 422. Like the circuit 250 described above, the circuit 448 is illustratively an Arduino Nano (rev. 3.0) circuit. As shown in FIG. 13, the automatic needle retraction mechanism 422 illustratively includes a DC needle retraction motor 450 that is coupled to the shaft 428 (and hence the needle tip 418). The motor 450 is positioned in the handle 424 of the surgical instrument 410 and may be, for example, an Uxcell 3 VDC micromotor. It should be appreciated that the motor 450 may be coupled to the shaft 428 by any appropriate means such as, for example, cam, rack and pinion, etc.

The motor 450 is coupled across the battery 252 of the circuitry 432. The collector-emitter path of a transistor 452, such as, for example, a BC547 transistor, is coupled in series with motor 450 across the battery 252.

The base of transistor 452 is coupled to the D7 terminal of the circuit 448. The motor 450 is thus controlled by the signal on the D7 terminal of circuit 448.

The circuitry 432 also includes the LED 230, which is coupled to the cathode of battery 252 and to the D2 terminal of circuit 448 through a 220 Ω resistor. In this embodiment, when the capacitance sensed by conductor plate 440 experiences a “step” change, indicating, for example, that the needle tip 418 has penetrated a target lumen such as the trachea, the circuit 432 is programmed to switch the D2 terminal “high,” thereby turning LED 230 “on.” Additionally, the circuit 432 is programmed to switch the terminal D7 “high,” energizing the motor 450 to retract the shaft 428 to reduce the likelihood of damage to the opposite wall of the trachea and to tissue beyond the opposite wall of the trachea.

In use, the needle tip 418 of the surgical instrument 410 may be used to form a puncture in the patient's tissue. As the needle tip 418 is advanced through the neck, the conductor plate 440 is engaged with the patient's tissue. When needle tip 418 reaches, and protrudes into, the target lumen of the patient, the conductor plate 440 is no longer in contact with the patient's tissue, thereby causing the capacitance to decrease sharply, and the circuit 448 switches the D2 terminal and D7 terminal “high,” thereby turning LED 230 “on” and energizing the motor 450 to retract the needle tip 418. In the illustrative embodiment, the needle tip 418 is retracted about 2-3 millimeters. After a predetermined period of time has elapsed, the motor 450 may be deenergized. A spring or other biasing element may be used to urge the needle tip 418 back into its forward position.

In other embodiments, the puncture instrument may be configured to locate and to differentiate between different masses (size and density) in the patient's body since different tissues have different capacitance signatures depending on their mass and density. For example, the puncture instrument may be configured to locate and/or differentiate between different organs and cancerous tissue such that the instrument may be used as probe or biopsy needle. In such embodiments, the instrument may activate a visual or audible in the instrument to alert the operator that the tip has exited one type of tissue and entered a different type of tissue.

In other embodiments, the puncture instrument may be configured to differentiate between good and bad fruit/vegetable based on its density. In such embodiments, the ripe fruit would have a different signature from that of an unripe or spoiled fruit/vegetable.

In other embodiments, a surgical instrument used to form a puncture in the patient's tissue may include a fiber optical thermometer to provide an indication to the user of the location of the needle tip. A fiber optical thermometer needle sensor can be used in electromagnetically strongly influenced environment, in microwave fields, power plants or explosion- proof areas and wherever measurement with electrical temperature sensors is not possible. The thermometer can have 1-255 channels for temperature measurement.

The fiber optical thermometer needle sensor typically consists of a (GaAs) semiconductor crystal that is mounted on the end of an optical fiber. Gallium arsenide (GaAs) is a compound of the elements gallium and arsenic. The needle tip and shaft may be completely non-metallic. The fiber optical sensor may be completely non-conductive and offers complete immunity to RFI, EMI, NMR and microwave radiation with high temperature operating capability, intrinsic safety, and non-invasive use.

The principle of operation is based on the temperature dependence of the band gap of GaAs. The GaAs crystal fixed on the tip of the fiber will be transparent at a wavelength above 850 nm. The position of the band edge is temperature dependent and is shifted about 0.4 nm/Kelvin. The light is directed via the optical fiber to the crystal, where it is absorbed and partially reflected back into the fiber. A miniature spectrometer provides a spectrum with the position of the band edge, from which the temperature is calculated. As the needle travels through different materials, it would give continuous feedback as to the temperature of the material that it is traveling through or when it enters a lumen or an inside space.

In other embodiments, a surgical instrument used to form a puncture in the patient's tissue may include an electromagnetic sensor to provide an indication to the user of the location of the needle tip. Electromagnetic fields, radio waves, microwaves and wireless signals are collectively referred to as radio frequency (RF) energy. Electromagnetic waves are measured by wavelength and frequency. Wavelength is the distance covered by one complete cycle of the electromagnetic wave. Frequency is the number of electromagnetic waves in one second, also known as a hertz or Hz. One Hz equals one cycle per second. One megahertz (MHz) equals one million cycles per second.

The needle can be used to transmit or receive electromagnetic waves and depending on the density of the material that the needle is traveling through the strength of the waves being transmitted or received will very. The material that the needle is traveling through insulates the electromagnetic waves and thus materials of different insulating factors will directly influence the strength of the radio waves as they pass through them.

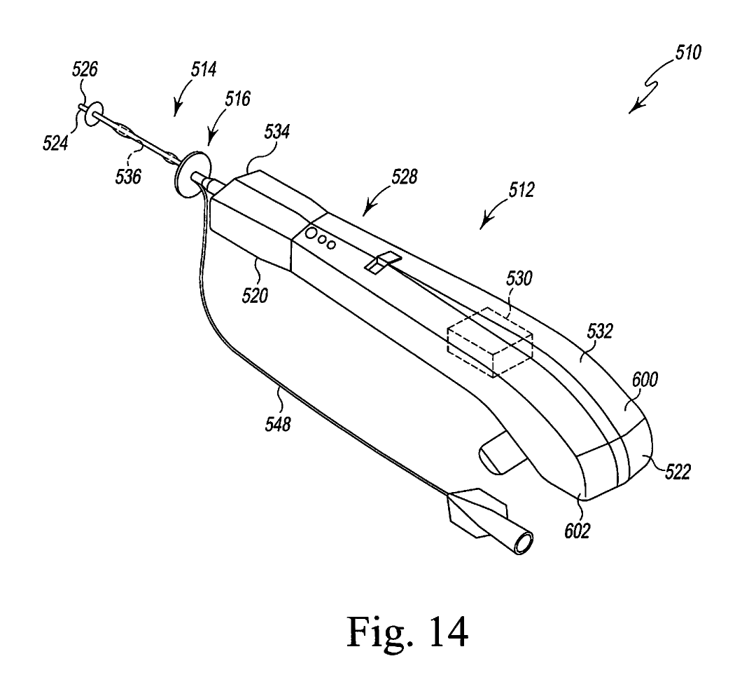

Referring now to FIG. 14, an instrument system 510 for forming and dilating an opening in a patient's tissue is shown. The instrument system 510 includes a puncture instrument 512 similar to the puncture instruments described above in regard to FIGS. 1-13 and a balloon catheter 514 that is removably coupled to the puncture instrument. The system 510 also includes a moveable retainer 516 that is positionable on the balloon catheter 514 to inhibit movement of the balloon catheter 514 during a surgical procedure, as described in greater detail below. The instrument system 510 may be used, for example, to create a puncture or incision in a tracheal wall of a patient and dilate the incision to receive a prosthesis such as, for example, a tracheostomy tube to form an air passageway for the patient.

Illustratively, the puncture instrument 512 may be used to form a puncture between the skin of the neck and the anterior wall of the trachea of a patient, but it should be appreciated that the puncture instrument 512 may be used to form other punctures, incisions, or openings in the patient's tissue. As shown in FIG. 14, the puncture instrument 512 includes an elongated body 520 having a proximal end 522 and a distal end 524. A needle tip 526 configured to pierce the tissue is formed at the distal end 524 of the body 520. As described in greater detail below, the puncture instrument 512 also includes an indicator 528 configured to notify a user that the needle tip 526 has penetrated the tissue and an automatic needle retraction mechanism 530 operable quickly to retract the needle tip 526 a short distance after the needle tip 526 has penetrated the tissue.

The elongated body 520 includes a handle 532 extending from the proximal end 522 to a distal handle end 534. A shaft 536 extends distally away from the handle 532 to the needle tip 526. In the illustrated embodiment, the shaft 536 is a cannula formed from a metallic material. In other embodiments, the shaft may be formed from a ceramic or plastic material. The needle tip 526 and the shaft 536 are integral, but it should be appreciated that in other embodiments the needle tip 526 and the shaft 536 may be formed as separate components and assembled.

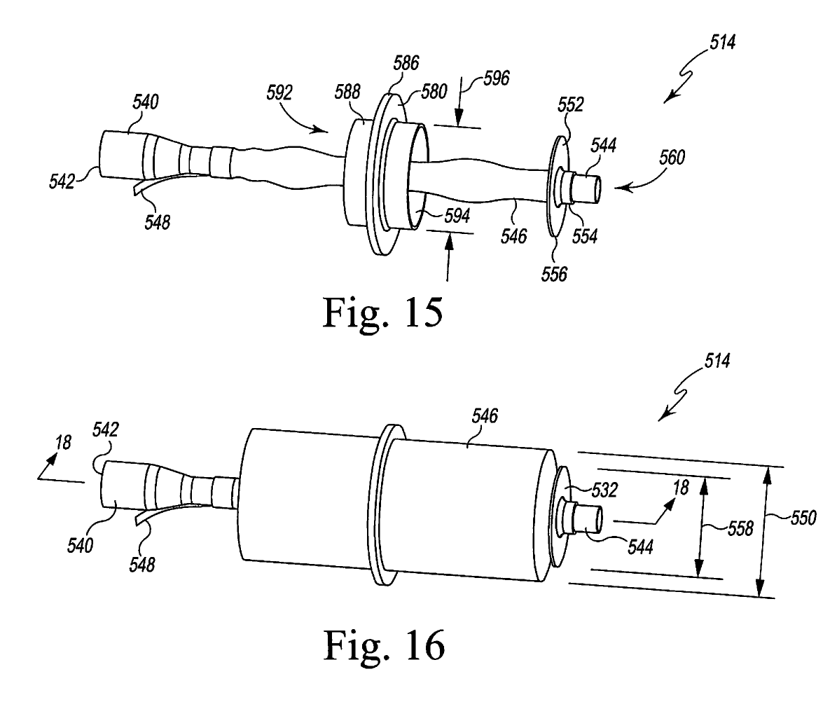

Referring now to FIGS. 15-16, the balloon catheter 514 includes a sheath 540 that extends from a proximal end 542 to a distal end 544. An inflatable balloon 546 extends axially along the sheath 540 between the ends 542, 544. The balloon 546 is illustratively formed from polyethylene terephthalate (“PET”). In other embodiments, the balloon may be formed from nylon, polyurethane, or other non-compliant material. The balloon catheter 514 also includes a tube 548 for the introduction of an inflating fluid into, and exhaust of inflating fluid from, the balloon 546. In the illustrative embodiment, the inflating fluid is water, but it should be appreciated that in other embodiments other suitable fluids may be used. When inflated, the balloon 546 has a generally cylindrical shape, as shown in FIG. 15, having an outer diameter 550 equal to about 0.65 inch (about 16.5 mm) in the illustrative embodiment. The outer diameter 550 is selected based on the desired size of the dilated incision and hence illustratively corresponds to the size of the desired prosthesis. In the illustrative embodiment, the rated pressure of the balloon 546 is about 16 atmospheres.

The balloon catheter 514 also includes a flange 552 that extends outwardly adjacent the distal end 544 of the sheath 540. The flange 552 include a collar 554 that is secured to the sheath 540 and a disk 556 that extends outwardly from the collar 554. The flange 552 is formed as a single, integral component from, for example, PET. In other embodiments, the flange may be formed from nylon, polyurethane, or other non-compliant material. Illustratively, the flange 552 has an outer diameter 558 that is less than the outer diameter 550 of the fully inflated balloon 546 but is greater than the diameter of the completely deflated balloon 546. As described in greater detail below, the outer diameter 558 of the flange 552 is selected such that the flange 552 may initially engage an inner surface of the patient's tissue to resist movement of the balloon catheter 514 during the steps of a surgical procedure. In the illustrative embodiment, the outer diameter 558 is equal to about 0.49 inch (about 12.4 mm). The disk 556 of the flange 552 is sized to deflect or deform relative to the sheath 540 to permit the flange 552 to pass through the opening defined by the puncture instrument 512, as described in greater detail below.

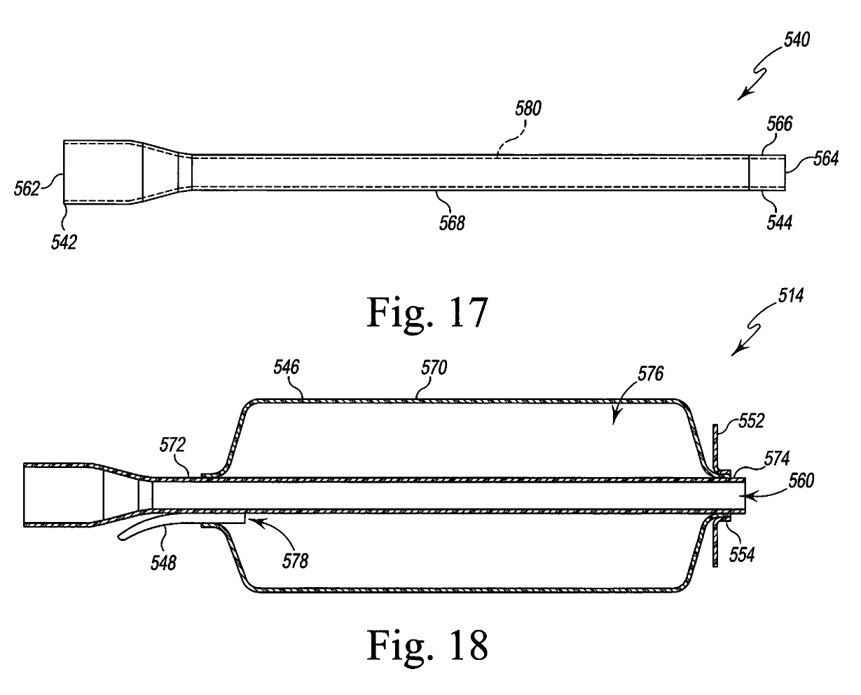

Referring now to FIG. 17, the sheath 540 of the balloon catheter 514 has a lumen 560 extending between an opening 562 at the proximal end 542 and another opening 564 defined the distal end 544. In the illustrative embodiment, the proximal end 542 of the sheath 540 is flared and the lumen 560 is enlarged to receive the needle tip 526 of the puncture instrument 512. The sheath 540 includes a tip 566 at its distal end 544 and an elongated body 568 that extends between the tip 566 and the proximal end 542. In the illustrative embodiment, the tip 566 and the elongated body 568 are formed as separate components that are then assembled to form the sheath 540. In the illustrative embodiment, the tip 566 and the body 568 are formed from plastic materials, but the body 568 is formed from a material that is harder than the tip 566. Illustratively, the tip 566 is formed from polyether block amide (Pebax®) having a hardness of a range of 30 durometer to 50 durometer. The elongated body 68 is also illustratively formed from Pebax® and has a hardness in a range of 70 durometer to 80 durometer. In one illustrative embodiment, the tip 566 has a hardness of 40 durometer, and the body 568 has a hardness of 72 durometer.

Referring now to FIG. 18, the balloon 546 has an inflatable central section 570 formed between a proximal neck 572 and a distal neck 574. The central section 570 defines a chamber 576 into which fluid may be introduced to inflate the central section 570. The distal neck 574 is oriented between the collar 554 of the flange 552 and the sheath 540, and the collar 554 and the neck 574 are bonded to the sheath 540 using an adhesive. The proximal neck 572 is bonded to the elongated body 568 of the sheath 540 also using an adhesive. In the illustrative embodiment, the adhesive is Dymex Adhesive 204-CTH-F. It should be appreciated that in other embodiments other adhesives may be used. As shown in FIG. 18, the tube 548 extends through the proximal neck 572 to an open end 578 oriented in the chamber 576.

Returning to FIG. 15, the system 510 also includes a moveable retainer 516 that is positionable lengthwise on the balloon 546 to resist movement of the balloon catheter 514 during a surgical procedure, as described above. The moveable retainer 516 is illustratively formed as a single, integral component from silicone rubber. It should be appreciated that in other embodiments movable retainer 516 may be formed from other elastomeric materials. Further, in other embodiments, the retainer 516 may be reinforced with a metallic web or frame. In the illustrative embodiment, the silicone rubber has a hardness in the range of 60 and 80 durometer. The retainer 516 has an annular body 580 that extends from a proximal end 582 to a distal end 584. The annular body 580 includes a flange 586 that extends outwardly from a proximal collar 588 and a distal collar 590. A passageway 592 extends from the end 582 to the end 584. The body 580 includes an inner cylindrical surface 594 that defines the passageway 592 and an inner diameter 596. In the illustrative embodiment, the inner diameter 596 is less than the outer diameter 558 of the balloon 546 such that when the balloon 546 is inflated, the retainer 516 fits snuggly onto the balloon 546 and is maintained in its position relative to the balloon 546. Illustratively, the inner diameter 596 of the retainer 516 is about 0.50 inch (about 12.7 mm). In the illustrative embodiment, the flange 586 defines an outer diameter of about 0.80 inch (about 20.3 mm).

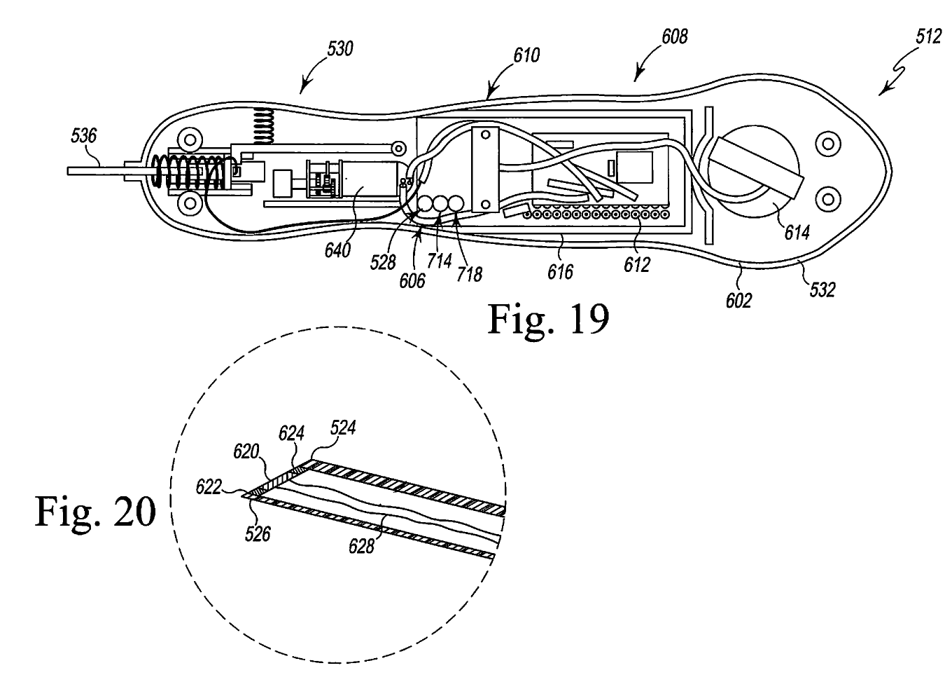

As described above, the dilation system 510 includes a puncture instrument 512 similar to the instruments 10, 210, 310 410 described above. As shown in FIG. 19, the instrument 512 includes a handle 532 and a shaft 536 that extends distally away from the handle 532. The handle 532 illustratively includes an upper housing 600 (see FIG. 14) that is configured to be coupled to a lower housing 602. As illustrated in FIG. 19, the indicator 528 includes a light source such as, for example, a light emitting diode (LED) 606 that is illustratively visible through an opening in the upper housing 600. The housings 600, 602 cooperate to define a chamber in which other electrical circuitry 608 is positioned. The circuitry 608 is operable to energize the LED 606 to provide a visual output to the user. In other embodiments, the indicator 528 may include other electrical circuitry to provide an audible output to the user. The puncture instrument 512 also includes a power switch 610, which is operable to supply power to the electrical circuitry 608 including LED 606.

As illustrated in FIG. 19, the power switch 610 and LED 606 are mounted on a circuit board 612 that is positioned in the handle 532. The circuit board 612 is electrically connected to a battery pack 614 positioned at one end of the handle 532 and the automatic needle retraction mechanism 530, which, as described above, is operable to retract the needle tip 526 a short distance after the needle tip 526 has penetrated the tissue. A metallic plate 616 is positioned below the circuit board 612 between the board 612 and the lower housing 602. In the illustrative embodiment, the plate 616 is formed from copper and is configured to provide a ground plane for the electrical circuitry 608, which makes the user the ground for the electrical circuitry.

As shown in FIG. 20, the electrical circuitry 608 also includes a conductor plate 620 that is positioned in the distal opening 622 of the needle tip 526. In the illustrative embodiment, the plate 620 is electrically insulated from the needle tip 526 by a non-conductive film 624. In other embodiments, the needle tip and/or needle shaft may be formed from a non-conductive material such as, for example, ceramic or plastic to electrically insulate the plate. The plate 620 and the film 624 cooperate to cover the opening 622 such that fluid is prevented from entering the needle tip 526. A wire or conductor 628 connects the backside of the plate 620 to the electrical circuitry 608. When a patient's tissue contacts the conductor plate 620, the electrical circuitry 608 is operable to detect the change in electrical capacitance caused by the contact with the tissue, as described in greater detail below.

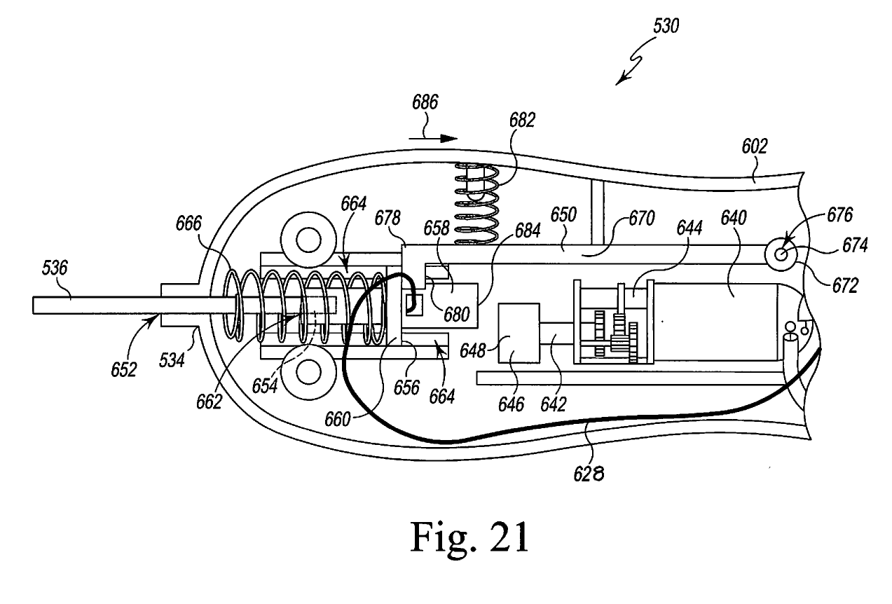

As described above, the instrument 512 includes an automatic needle retraction mechanism 530 operable to retract the needle tip 526 a short distance after the needle tip 526 has penetrated the tissue. As shown in FIG. 21, the needle retraction mechanism 530 includes an electric motor 640 that is connected to an output shaft 642 via a gearbox 644. The needle retraction mechanism 530 also includes a cam 646 that is connected to the output shaft 642. The cam 646 illustratively includes an oblong curved outer surface 648 such that when the cam 646 is rotated by the output shaft 642, the outer surface 648 is moved into and out of engagement with a locking arm 650 that maintains the needle shaft 536 in an extended position.

The needle shaft 536 extends through an opening 652 defined in the distal handle end 534, and the shaft 536 includes a proximal end 654 that is secured to a mounting bracket 656 positioned in the handle 532. The mounting bracket 656 includes a cylindrical body 658 and a slide plate 660 that extends outwardly from the body 658. An aperture 662 is defined at one end of the cylindrical body 658, which receives the proximal end 654 of the shaft 536 and provides a passageway through which the connecting wire 628 passes to connect the conductor plate 620 to the other electrical circuitry 608.

As shown in FIG. 21, the edges of the slide plate 660 are received in a pair of guide slots 664 defined in the handle 532, which guide the movement of the mounting bracket 656 as the needle tip 526 is retracted. A biasing element such as, for example, a spring 666 positioned between the slide plate 660 and the distal handle end 534. In the illustrative embodiment, the spring 666 is configured to bias the slide plate 660 away from the distal handle end 534 and hence bias the needle tip 526 is the retracted position.

The locking arm 650 of the automatic needle retraction mechanism 530 includes an elongated shaft 670 pivotally coupled to the handle 532. A pivot pin 674 extends outwardly from the lower housing 602 and is received in a bore 676 defined in an end 672 of the elongated shaft 670. The elongated shaft 670 extends from the end 672 to an opposite end 678 positioned adjacent the mounting bracket 656. The locking arm 650 includes a tip 680 that extends from the end 678 toward the mounting bracket 656, as illustrated in FIG. 21. The retraction mechanism 530 also includes another biasing element or spring 682 positioned between the elongated shaft 670 and an inner wall of the handle 532.

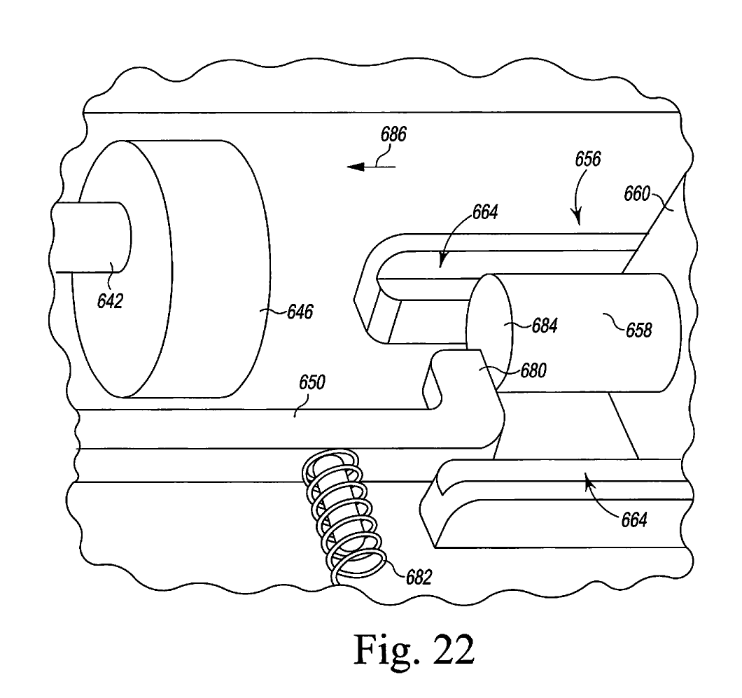

When the needle shaft 536 is in its extended position and ready for insertion into a patient's tissue, the locking arm 650 may initially be engaged with a proximal end 684 of the mounting bracket 656, as shown in FIG. 22. The spring 682 applies a force to the elongated shaft 670 to bias the locking arm 650 in engagement with the proximal end 684, thereby resisting the force exerted by the spring 666 against the slide plate 660 and maintaining the needle shaft 536 in the extended position.

As described above, the automatic needle retraction mechanism 530 is operable to quickly retract the needle tip 526 a short distance after the needle tip 526 has penetrated the tissue. To do so, the motor 640 is energized to rotate the output shaft 642 and the cam 646. As the cam 646 is rotated, its oblong curved outer surface 648 is advanced into engagement with the elongated shaft 670 to move the shaft 670 (and hence the locking arm 650) away from the proximal end 684 of the mounting bracket 656. When the locking arm 650 disengages from the mounting bracket 656, the spring 666 urges the mounting bracket 656 in the direction indicated by arrow 686 in FIGS. 21-22. As the mounting bracket 656 moves, the needle tip 526 retracts away from the opposite wall of the patient's lumen. The spring 666 urges the bracket 656 to continue to move as indicated by arrow 686 until the slide plate 660 is advanced into contact with the locking arm tip 680, as shown in FIG. 21.

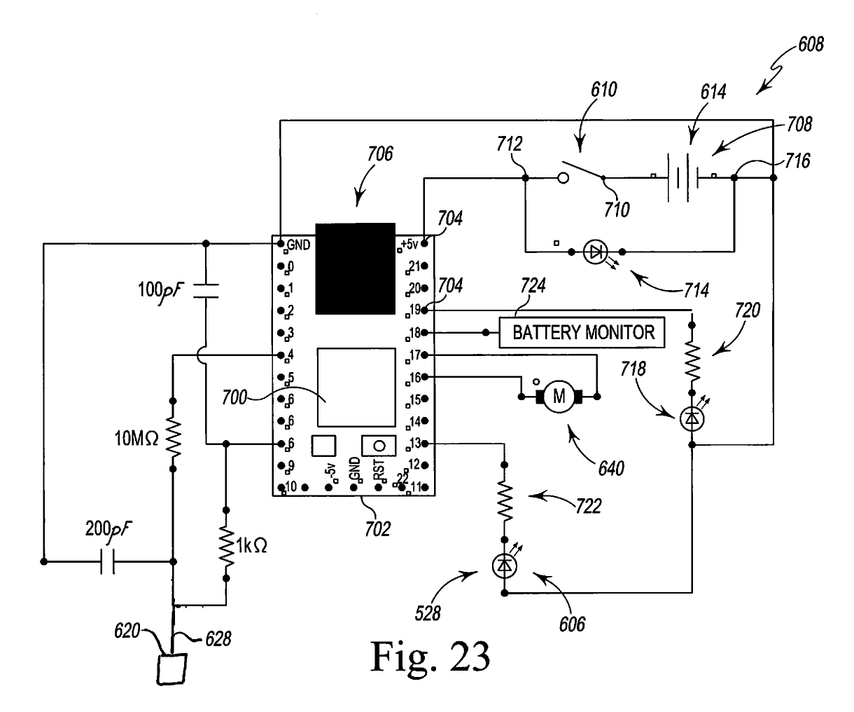

Referring now to FIG. 23, the electrical circuitry 608 is illustrated in greater detail. The schematic and block circuit diagram descriptions that follow identify specific integrated circuits and other components of the circuitry 608 and in many cases specific sources for these. Specific terminal and pin names and numbers are generally given in connection with these for the purposes of completeness. It is to be understood that these terminal and pin identifiers are provided for these specifically identified components. It is to be understood that this does not constitute a representation, nor should any such representation be inferred, that the specific components, component values or sources are the only components available from the same or any other sources capable of performing the necessary functions. It is further to be understood that other suitable components available from the same or different sources may not use the same terminal/pin identifiers as those provided in this description.

The circuitry 608 includes a microprocessor 700 such as, for example, an 8-Bit AVR 16 MHz Processor (ATMEGA32U4) commercially available from Atmel Corporation. As shown in FIG. 23, the microprocessor 700 is attached a circuit 702 that also includes various terminals 704 connected to other circuitry 608. An I/O port 706 such as, for example, a USB port, is attached to the circuit 702 to permit a user to upload software and data to, and download from, the microprocessor 700. Illustratively, the microprocessor 700, the circuit 702, and the I/O port 706 are available in a Teensy 2.0 USB-based microcontroller development system by PJRC. A voltage supply includes two 3 VDC batteries 708 in the battery pack 614, the anode of which is coupled to one terminal 710 of the power switch 610. The other terminal 712 of switch 610 is coupled to the 5V terminal of the circuit 702 and to the anode of a “Power Indicator” LED 714. The cathode of the Power Indicator LED 714 is coupled to the cathode of the battery pack 614 and to the GrouND terminal of the circuit 702 at the terminal 716.

The circuitry 608 also includes a “Low Battery” LED 718, which is energized by the microprocessor 700 when battery voltage drops below a predetermined threshold. The cathode of the LED 718 is connected through a 220 Ω resistor 720 to the “19” terminal of the circuit 702. The anode of the LED 718 is connected to the GrouND terminal of the circuit 702 and an anode of the LED 528 of the indicator 606. The cathode of the LED 528 is connected to the “13” terminal of the circuit 702 through another 220 Ω resistor 722.

As shown in FIG. 23, the motor 640 is connected to the “16” and “17” terminals, while a battery monitor 724 is connected to the “18” terminal of the circuit 702. The conductor plate 620 of the needle tip 526 is coupled via the wire 628 through a 10 MΩ resistor to the “4” terminal of the circuit 702. The wire 628 (and hence the “4” terminal) is also connected to the ground terminal of the circuit 702 via a 220 pF capacitor, which assists in stabilizing the circuit. The conductor plate 620 is also coupled via the wire 628 through a 1 KΩ resistor to the “8” terminal of the circuit 620. The wire 628 (and hence the “8” terminal) is connected to the ground terminal of the circuit 702 via a 100 pF capacitor, which also assists in stabilizing the circuit.

Illustratively, when the capacitance sensed by microprocessor 700 through the conductor plate 620 experiences a “step” change, indicating, for example, that the needle tip 526 has penetrated a lumen, the microprocessor 700 is programmed to switch the “13” terminal continuously “high,” thereby turning the indicator LED 528 continuously “on.” Additionally, the microprocessor 700 is programmed to switch the “16” terminal to “high,” thereby energizing the motor 640 to retract the needle tip 526 and reduce the likelihood of damage to the opposite wall of the trachea and to tissue beyond the opposite wall of the trachea.

In use, the needle tip 526 of the puncture instrument 512 may be used to form a puncture in the patient's tissue. To do so, the needle tip 526 may be first inserted into the lumen 560 of the balloon catheter 514 such that the tip 526 extends outwardly from the distal opening 564. When the user toggles the power switch 610, power is supplied to the circuit 702 and to the LED 714, which is energized to indicate that power is “on.” The microprocessor 700 monitors the signal on the “18” terminal from the battery monitor 724. If the microprocessor 700 determines that the voltage signal on the “18” terminal is below a predetermined threshold, the microprocessor 700 is programmed to switch the “19” terminal to “high,” thereby turning the indicator LED 718 “on” to indicate to the user that the battery pack 614 should be replaced.

If the microprocessor 700 determines the voltage signal is above the predetermined threshold, the needle tip 526 may be advanced into contact with the patient's tissue, illustratively the patient's neck tissue. When the tip 526 engages the patient's tissue, the capacitance experienced by conductor plate 620 increases sharply. In the illustrative embodiment, the microprocessor 700 is programmed to consecutively toggle the “13” terminal “high” and “low,” thereby causing the LED 528 to flash “on” and “off” to indicate to the user that the instrument 512 is armed.

As the needle tip 526 is advanced through the neck, the conductor plate 620 remains engaged with the patient's tissue. When needle tip 526 reaches, and protrudes into, the target lumen, the conductor plate 620 is no longer in contact with the patient's tissue, thereby causing the capacitance to decrease sharply, and the microprocessor 700 is programmed to switch the “13” terminal continuously “high,” thereby turning the indicator LED 528 continuously “on.” Additionally, the microprocessor 700 is programmed to switch the “16” terminal to “high,” thereby energizing the motor 640 to retract the needle tip 526. When the motor 640 is energized, the output shaft 642 and hence the cam 646 are rotated. As the cam 646 is rotated, its oblong curved outer surface 648 is advanced into engagement with the elongated shaft 670 to move the shaft 670 (and hence the locking arm 650) away from the proximal end 684 of the mounting bracket 656. When the locking arm 650 disengages from the mounting bracket 656, the spring 666 urges the mounting bracket 656 in the direction indicated by arrow 686 in FIGS. 21-22. As the mounting bracket 656 moves, the needle tip 526 retracts away from the opposite wall of the patient's lumen. The spring 666 urges the bracket 656 to continue to move as indicated by arrow 686 until the slide plate 660 is advanced into contact with the locking arm tip 680, as shown in FIG. 21. In the illustrative embodiment, the needle tip 526 is retracted about 2-3 millimeters.

With the balloon catheter 514 positioned on the puncture instrument 512 and the balloon 546 deflated, the distal tip 566 of the sheath 540 and the flange 552 may be advanced through the incision made by the needle tip 526. As the flange 552 passes through the incision, it may be deflected to decrease its diameter and permit it to enter the incision. Once within the patient's trachea or other lumen, the flange 552 may deflect outward to its normal diameter 558. Because the tip 566 of the sheath 540 is formed from a relatively soft material, it may be advanced into contact with the opposite wall of the patient's lumen without fear of damage. When the flange 552 is positioned in the patient's lumen, the balloon catheter 514 may be pulled outward to advance the flange 552 into contact with an inner surface of the lumen.

With the flange 552 engaged with the inner surface of the trachea, the retainer 516 may be advanced along deflated balloon 546 to engage the outer surface of the trachea opposite the flange 552. In that way, the flange 552 and the retainer 516 cooperate to maintain the balloon 546 in position relative to the patient's tissue and inhibit movement of the balloon 546. The central section 570 of the balloon 546 may then be inflated to dilate the incision to the desired size.

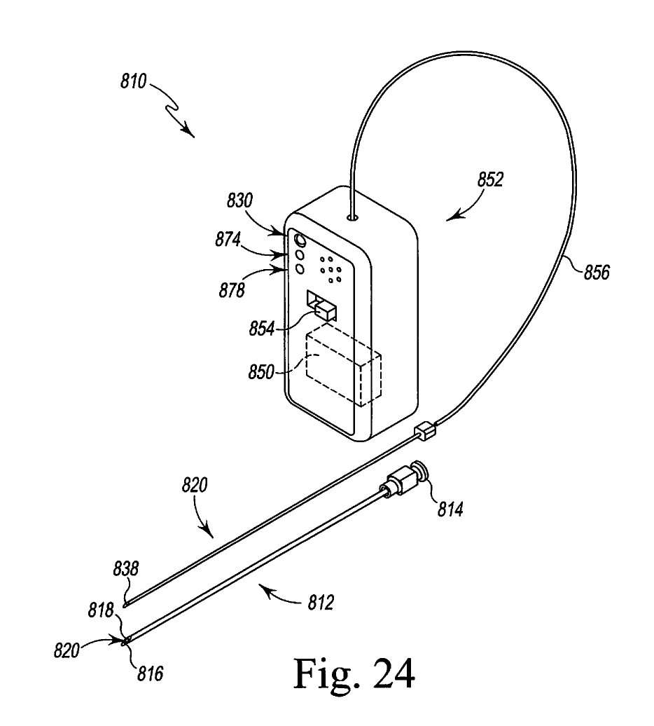

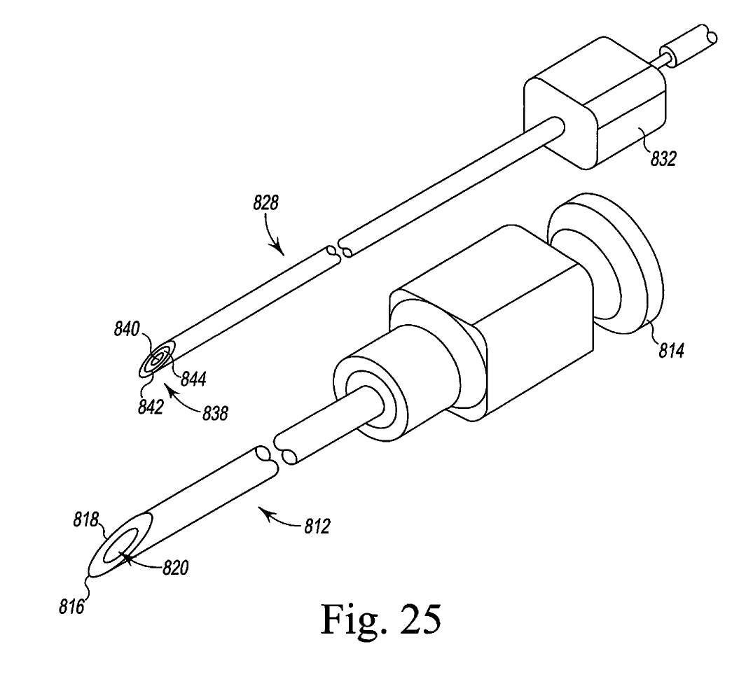

Referring now to FIGS. 24-26, another surgical instrument system 810 configured for insertion into the soft tissue of a patient is illustrated. The surgical instrument system 810 includes an elongated needle body 812 that extends from a proximal end 814 to a distal end 816. A needle tip 818 configured to pierce the tissue is formed at the distal end 816 of the body 812. The needle body 812 has a lumen or passageway 820 extending through the ends 814, 816, as shown in FIG. 25. In the illustrative embodiment, a catheter may be inserted into the passageway 820 to provide, for example, epidural anesthesia, to a patient. The surgical instrument system 810 also includes a probe 828 that is sized to be positioned in the passageway 820 of the needle 812. The probe 828 is connected to an indicator 830 that is configured to notify a user that the needle tip 818 has penetrated the tissue, as described in greater detail below.

The probe 828 includes a base 832 and a shaft 836 that extends distally away from the base 832 to a tip 838. In the illustrated embodiment, the shaft 836 is a cannula formed from an electrically conductive material. The tip 838 and the shaft 836 are integral, but it should be appreciated that in other embodiments the tip 838 and the shaft 836 may be formed as separate components and assembled. As shown in FIG. 25, the probe 828 includes a conductor plate 840 that is positioned in the distal opening 842 of the tip 838. In the illustrative embodiment, the plate 840 is electrically insulated from the tip 838 by a non-conductive film 844. In other embodiments, the shaft may be formed from a non-conductive material such as ceramic or plastic to insulate the plate. The plate 840 and the film 844 cooperate to cover the opening 842 such that fluid is prevented from entering the tip 838. When a patient's tissue contacts the conductor plate 840, electrical circuitry 850 of the system 810 is operable to detect the change in electrical capacitance caused by the contact with the tissue, as described in greater detail below.

Returning to FIG. 24, the system 810 includes a control box 852 that houses the electrical circuitry 850, including the indicator 830. In the illustrative embodiment, the control box 852 has a power switch 854 that may be toggled to energize the electrical circuitry 850. A cable 856 connects the electrical circuitry 850 with the probe 828.

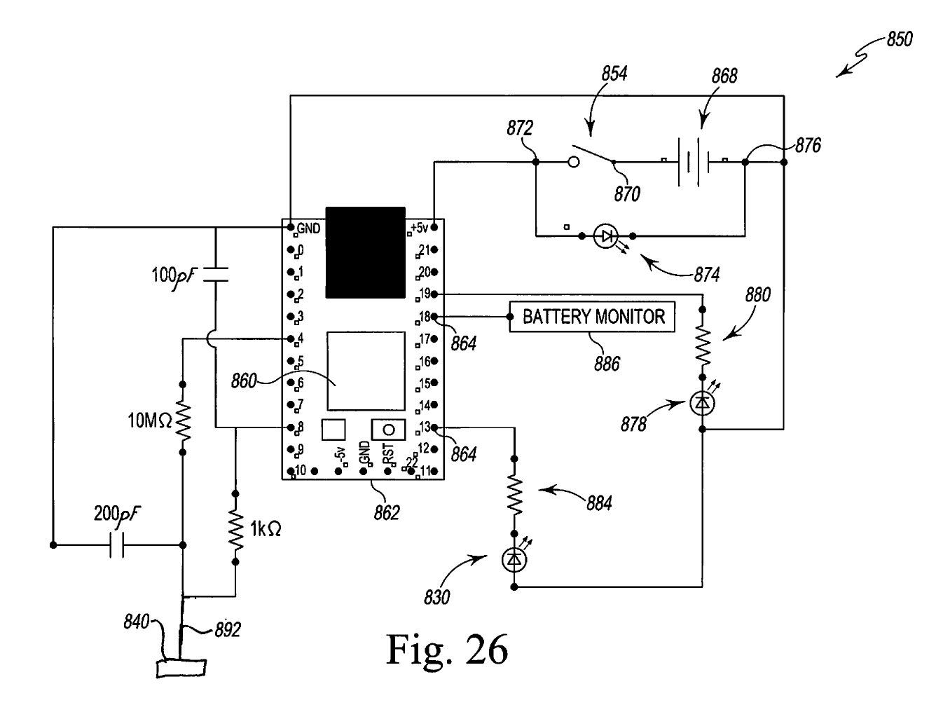

Referring now to FIG. 26, the circuitry 850 includes a microprocessor 860 such as, for example, an 8-Bit AVR 16 MHz Processor (ATMEGA32U4) commercially available from Atmel Corporation. The microprocessor 860 is attached a circuit 862 that also includes various terminals 864 connected to other circuitry 850. An I/O port 866 such as, for example, a USB port, is attached to the circuit 862 to permit a user to upload software and data to, and download from, the microprocessor 860. Illustratively, the microprocessor 700, the circuit 702, and the I/O port 706 are available in a Teensy 2.0 USB-based microcontroller development system. A voltage supply includes two 3 VDC batteries 868, the anodes of which are coupled to one terminal 870 of the power switch 854. The other terminal 872 of switch 854 is coupled to the 5V terminal of the circuit 862 and to the anode of a “Power Indicator” LED 874. The cathode of the Power Indicator LED 874 is coupled to the cathodes of the batteries 868 and to the GrouND terminal of the circuit 862 at the terminal 876.

The circuitry 850 also includes a “Low Battery” LED 878, which is energized by the microprocessor 860 when battery voltage drops below a predetermined threshold. The cathode of the LED 878 is connected through a 220 Ω resistor 880 to the “19” terminal of the circuit 862. The anode of the LED 878 is connected to the GrouND terminal of the circuit 862 and an anode of the indicator LED 830. The cathode of the LED 830 is connected to the “13” terminal of the circuit 862 through another 220 Ω resistor 884. A battery monitor 886 is connected to the “18” terminal of the circuit 862.

The conductor plate 840 of the tip 838 is coupled via a wire 892 through a 10 MΩ resistor to the “4” terminal of the circuit 862. The wire 892 (and hence the “4” terminal) is also connected to the ground terminal of the circuit 862 via a 220 pF capacitor, which assists in stabilizing the circuit. The conductor plate 840 is also coupled via the wire 892 through a 1 KΩ resistor to the “8” terminal of the circuit 862. The wire 892 (and hence the “8” terminal) is connected to the ground terminal of the circuit 862 via a 100 pF capacitor, which also assists in stabilizing the circuit.

Illustratively, when the capacitance sensed by microprocessor 860 through the conductor plate 840 experiences a “step” change, indicating, for example, that the tip 838 has penetrated a lumen, the microprocessor 860 is programmed to switch the “13” terminal continuously “high,” thereby turning the indicator LED 830 continuously “on.”

In use, the instrument system 810 may be used to form a puncture in the patient's tissue. To do so, the probe 828 may be first inserted into the lumen 820 of the needle 812 such that the probe tip 838 is exposed at the needle tip 818. When the user toggles the power switch 854, power is supplied to the circuitry 850 and to the LED 874, which is energized to indicate that power is “on.” The microprocessor 860 monitors the signal on the “18” terminal from the battery monitor 886. If the microprocessor 860 determines that the voltage signal on the “18” terminal is below a predetermined threshold, the microprocessor 860 is programmed to switch the “19” terminal to “high,” thereby turning the indicator LED 878 “on” to indicate to the user that the batteries 868 should be replaced.

If the microprocessor 860 determines the voltage signal is above the predetermined threshold, the probe tip 838 and needle tip 818 may be advanced into contact with the patient's tissue, illustratively the skin covering a patient's spinal column. When the probe tip 838 engages the patient's tissue, the capacitance experienced by conductor plate 840 increases sharply. In the illustrative embodiment, the microprocessor 860 is programmed to consecutively toggle the “13” terminal “high” and “low,” thereby causing the LED 830 to flash “on” and “off” to indicate to the user that the instrument system 810 is armed. As the needle 812 (and hence the probe 838) is advanced into the spinal column, the conductor plate 840 remains engaged with the patient's tissue.

When the probe tip 838 reaches, and protrudes into, the target lumen (e.g., the interior of the spinal column), the capacitance on the conductor plate 840 decreases sharply, and the microprocessor 860 is programmed to switch the “13” terminal continuously “high,” thereby turning the indicator LED 830 continuously “on” to inform the user to hold the needle 812 in position. The user may then remove the probe 828 from the lumen 820 of the needle 812 while leaving the needle 812 inserted into the patient's tissue. The user may then use the lumen 820 to position, for example, a catheter to provide fluids to the patient.

It should be appreciated that although the concept of detecting a lumen in a patient's body has been described above in reference to surgical instruments that may be used to create punctures in a patient's tissue, the techniques and concepts described above may be incorporated into other surgical instruments such that entry into a lumen or movement between various tissue types may be detected. For example, any surgical cutting tool such as, for example, a cutting blade, reamer, drill, or other instrument may include circuitry to detect fluctuating levels of electrical capacitance and thereby determine when a distal end of the cutting tool has entered a lumen. Other surgical instruments such as, for example, guides, trials, probes, and so forth may also include circuitry to detect fluctuating levels of electrical capacitance and thereby determine when a distal end of the surgical instrument has entered a lumen.

While the disclosure has been illustrated and described in detail in the drawings and foregoing description, such illustration and description is to be considered as exemplary and not restrictive in character, it being understood that only illustrative embodiments have been illustrated and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

What is claimed is:

1. A dilation instrument system comprising:
a balloon catheter configured to be positioned in an opening defined in a tracheal wall of a patient, the catheter including a sheath having a proximal end and a distal end, an inflatable balloon extending over the sheath between the proximal end and the distal end, and a deflectable retention flange secured to the distal end of the sheath,
a retainer positioned over the balloon, the retainer being configured to move relative to the balloon such that upon inflation of the balloon when the balloon is positioned in the opening in the tracheal wall, the retainer engages the tracheal wall to inhibit movement of the balloon catheter, and
a surgical instrument configured to be coupled to the balloon catheter, the surgical instrument comprising (i) an elongated shaft sized to be positioned in a lumen defined in the sheath, (ii) a needle tip configured to puncture the tracheal wall, the needle tip being configured to extend outwardly from the distal end of the sheath when the surgical instrument is coupled to the balloon catheter, and (iii) a visual indicator operable to be energized to indicate that the needle tip is positioned in the lumen of the patient's trachea.

2. The dilation instrument system of claim 1, wherein:
the inflatable balloon has a maximum diameter when inflated, and
the retainer includes an annular body having an inner diameter that is less than the maximum diameter of the inflatable balloon.

3. The dilation instrument system of claim 2, wherein the annular body includes a first collar extending in a first direction, a second collar extending outwardly in a second direction opposite the first direction, and a passageway extends between an opening defined in the first collar and an opening defined in the second collar, the passageway defining the inner diameter of the annular body.

4. The dilation instrument system of claim 1, wherein the retainer is formed from an elastomeric material.

5. The dilation instrument system of claim 4, wherein the elastomeric material is a liquid silicone rubber having hardness in a range of 60 and 80 durometer.

6. The dilation instrument system of claim 1, wherein the balloon is formed from a polymeric material.

7. The dilation instrument system of claim 6, wherein the polymeric material is selected from a group consisting of polyethylene, polyurethane, and nylon.

8. The dilation instrument system of claim 1, wherein the surgical instrument includes an electrical switch that is moveable between a closed position in which the visual indicator is energized, and an open position in which the visual indicator is de-energized.

9. The dilation instrument system of claim 8, wherein a first electrical contact of the electrical switch includes the needle tip.

10. The dilation instrument system of claim 9, further comprising a target body configured to be inserted into a passageway of the patient, the target body including an electrically conductive surface, a second electrical contact of the electrical switch including the electrically conductive surface such that when the needle tip engages the electrically conductive surface, the switch is closed to energize the visual indicator.

11. The dilation instrument system of claim 10, wherein the surgical instrument further comprises a retraction mechanism operable to automatically retract the needle tip in response to the closure of the switch.

12. A method for performing a surgical procedure comprises:
advancing a target body including an electrically conductive surface into an internal passageway of the patient,
inserting a needle tip of a surgical instrument into a first surface of a patient's tissue,
advancing the needle tip through the tissue,
monitoring an indicator of the surgical instrument while advancing the needle tip through the tissue,
contacting the electrically conductive surface of the target body to energize a light source of the indicator to indicate that the needle tip has penetrated a second surface of the tissue, and
withdrawing the needle tip from the tissue in response to the indicator indicating the needle tip has penetrated the second surface of the tissue.

13. The method of claim 12, wherein withdrawing the needle tip from the tissue comprises retracting the needle tip automatically in response to contacting the electrically conductive surface of the target body.

14. The method of claim 12, wherein contacting the electrically conductive surface of the target body comprises closing a circuit including the electrically conductive surface and the needle tip.

15. A surgical instrument system comprising an elongated body including a handle and a shaft extending from the handle to a distal end configured to pass through a patient's tissue, an indicator including a light source, a sensor operable to generate an electrical signal, and a control circuit configured to: (i) receive the electrical signal from the sensor, (ii) determine whether the distal end has penetrated a lumen of a patient, and (iii) energize the light source when the distal end has penetrated the lumen of the patient.

16. The surgical instrument system of claim 15, further comprising:
a target body configured to be inserted into a passageway of the patient, the target body including an electrically conductive surface sized to be positioned in the passageway,
wherein the sensor includes the electrically conductive surface and the distal end of the shaft that form a circuit that is closed by contact between the electrically conductive surface, the circuit being configured to generate an output signal when closed.

17. The surgical instrument system of claim 16, the system further comprising a retraction mechanism operable to retract the distal end, wherein the control circuit is configured receive the output signal from the circuit and energize the retraction mechanism in response to receiving the output signal.

18. The surgical instrument system of claim 17, wherein the retraction mechanism includes a biasing element configured to bias the distal end in a retracted position.

19. The surgical instrument system of claim 18, wherein the retraction mechanism includes a locking arm configured to maintain the distal end in an extended position.

20. The surgical instrument system of claim 19, wherein the retraction mechanism includes an actuator configured to move the locking arm to a disengaged position when the actuator is energized, and when the locking arm is in the disengaged position, the biasing element is configured to urge the distal end to move to the retracted position.

21. The surgical instrument system of claim 20, further comprising a percutaneous dilation balloon, a stationary deflectable retention flange, and a moveably positionable retainer, wherein the retainer is positioned over the balloon and is configured to move relative to the balloon such that upon inflation of the balloon when the balloon is positioned in an opening in a wall of the patient's tissue, the retainer is positioned adjacent to the wall to inhibit movement of the balloon catheter.
